(19) United States
(12) Patent Application Publication
(10) Pub. No.: US 2024/0148956 A1
(43) Pub. Date: May 9, 2024

(54) FILTER FOR MECHANICAL THROMBECTOMY DEVICE AND METHOD OF USING THE SAME

(71) Applicant: AngioDynamics, Inc., Latham, NY (US)

(72) Inventors: Kevin SWIFT, Hudson, MA (US); Mark Girard, Waltham, MA (US); Kenneth Leblanc, Pepperell, MA (US); Anthony Hien, Stoneham, MA (US); Damon Casiello, Worcester, MA (US)

(73) Assignee: AngioDynamics, Inc., Latham, NY (US)

(21) Appl. No.: 18/503,864

(22) Filed: Nov. 7, 2023

Related U.S. Application Data
(60) Provisional application No. 63/423,228, filed on Nov. 7, 2022.

(51) Int. Cl. A61M 1/00; A61B 17/22

(52) U.S. Cl. CPC A61M 1/79; A61B 17/22; A61B 2017/22079; A61M 2205/583

(57) ABSTRACT

Systems and methods are described for the filtering and reinfusion of blood during a thrombectomy procedure. An example of such a system includes a pump and a visualization device including a screen to filter thrombi from the blood. The visualization device is configured to be opened to allow the screen and filtered thrombi to be removed during the thrombectomy procedure.

### CROSS-REFERENCE TO RELATED APPLICATIONS

This application claims the benefit of priority to U.S. Provisional Application No. 63/423,228 entitled “Filter for Mechanical Thrombectomy Device,” filed Nov. 7, 2022, which is incorporated herein by reference in its entirety.

### BACKGROUND

Current manual mechanical thrombectomy devices on the market remove blood and undesired material and/or clot from a patient into a waste receptacle via collection tubing. One such device can be found in U.S. patent application Ser. No. 17/170,782, published as U.S. Patent Application Pub. No. 2021/0275199A1, titled DEVICE AND METHOD FOR MANUAL ASPIRATION AND REMOVAL OF AN UNDESIRABLE MATERIAL, filed Feb. 8, 2021, which is hereby incorporated by reference as if set forth in its entirety herein. Such a device includes a suction cannula/procedure sheath subassembly, a manually operated aspiration device, and a waste assembly comprising the waste receptacle and collection tubing that enables blood and undesired material received by the aspiration device to be transferred to the waste receptacle. There is not currently a way to filter the undesired material and/or clot from the blood and reinfuse filtered blood back into the patient with these manual mechanical thrombectomy devices. There exists a need for improved filtering of the blood from undesired material and/or clot and for reinfusion of the filtered blood back into the patient.

### SUMMARY

In one embodiment a method of filtering fluid from a patient comprises fluidly coupling a pump to a filter system including a first tube, a first reservoir, a second tube, and a visualization device including a first filter member; aspirating fluid from the patient using the pump wherein the fluid contains an undesired material; filtering the fluid using the first filter member to capture at least a portion of the undesired material; passing the filtered fluid to the reservoir; visualizing the undesirable material from the first filter member; and replacing the first filter member with a replacement filter member.

In one embodiment a method of filtering fluid from a patient comprises fluidly coupling a pump to a filter system including a first tube, a reinfusion cannula, a second tube, a visualization device including a first filter member, and a second filter member; aspirating fluid from the patient using the pump wherein the fluid contains an undesired material; filtering the fluid using the first filter member to capture at least a first portion of undesired material; filtering the fluid using the second filter member to capture a second portion of the undesired material, thereby generating a filtered fluid; reinfusing the filtered fluid into the patient using the reinfusion cannula; visualizing the undesirable material from the first filter member; and replacing the first filter member with a replacement filter member.

### BRIEF DESCRIPTION OF THE DRAWINGS

FIG. 1 depicts an illustrative system for filtering and passive reinfusion of blood in accordance with an embodiment.

FIG. 2 depicts an illustrative system for filtering and passive reinfusion of blood in accordance with an embodiment.

FIGS. 3A-3C depict an illustrative system of a visualization device in accordance with an embodiment.

FIG. 4 depicts an illustrative screen for filtering blood in accordance with an embodiment.

FIGS. 5-6 depict alternate illustrative systems for filtering and passive reinfusion of blood in accordance with various embodiments.

FIG. 7A-7C depict an illustrative system of a visualization device in accordance with an embodiment.

FIG. 8 depicts an illustrative screen for filtering blood in accordance with an embodiment.

FIGS. 9-12 depict alternate illustrative systems for filtering blood and active reinfusion of blood in accordance with various embodiments.

FIGS. 13A-13B depict an illustrative system for filtering blood in accordance with an embodiment.

FIG. 14 depicts an illustrative system for collecting filtered blood in accordance with an embodiment.

FIGS. 15-17 depict alternate illustrative systems for filtering blood in accordance with various embodiments.

FIG. 18 depicts an illustrative screen for filtering blood in accordance with an embodiment.

FIGS. 19-20 depict illustrative systems for filtering and passive reinfusion of blood in accordance with various embodiments.

FIG. 21 depicts a flow diagram of an illustrative method of filtering blood in accordance with an embodiment.

FIG. 22 depicts a flow diagram of an illustrative method of replacing a screen in a visualization device in accordance with an embodiment.

FIGS. 23-24 depict flow diagrams of illustrative methods of filtering blood and reinfusing the blood in accordance with various embodiments.

### DEFINITIONS

As used herein, the term “about” when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

As used herein, the term “undesired material” means a material that is detrimental to the health of a patient when present in the blood stream of a patient. For example, in some embodiments, an undesired material is a thrombus, clot, lesion, pulmonary embolism, tumors, infective vegetations, foreign bodies or other natural objects.

As used herein, the term “large bore thrombus” means a thrombus with a large volume. For example, in some embodiments, a large bore thrombus are thrombi able to be filtered by a 500 μm filter.

As used herein, the term “passive reinfusion” or “indirect reinfusion” refers to the process of filtering and/or removing undesirable material from an unfiltered fluid that has been aspirated or removed from a body The filtered fluid is housed or stored in a reservoir for a period of time before a clinician by separate means reinfuses the filtered fluid back to the body. For example, in some embodiments a passive reinfusion system comprises a reservoir or other storage member in fluid communication with the system to house or store filtered fluid for a period of time before the filtered fluid is reinfused or returned back to the body.

As used herein, the term “active reinfusion” or “direct reinfusion” refers to the process of filtering and/or removing undesirable material from an extracted bodily fluid (i.e., an unfiltered fluid) that is actively being removed or aspirated during a medical procedure. The filtered fluid is reinfused or reintroduced back into the body during the same medical procedure, or without being housed or stored in a reservoir for a period of time. For example, in some embodiments an active reinfusion system comprises a return cannula in fluid communication with the system to continuously reinfuse or reintroduce the filtered fluid back to the body. An active reinfusion system may comprise a closed looped system in which the aspiration catheter and return catheter are both in fluid communication and the filtered fluid is not stored in any reservoir or storage member for a period of time.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those skilled in the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### DETAILED DESCRIPTION

This disclosure is not limited to the particular systems, devices and methods described, as these may vary. The terminology used in the description is for the purpose of describing the particular versions or embodiments only and is not intended to limit the scope. The filtering devices, systems and methods disclosed herein are equally applicable to any mechanical thrombectomy devices without departing from aspects of the disclosure described herein.

### Systems

Systems may be assembled to aid in the filtering of fluid, such as blood, for a thrombectomy device. In some embodiments, the system comprises a pump, a filter device comprising a visualization device, and a reservoir. In some embodiments, the visualization device comprises a first filter member. In some embodiments, the visualization device is configured to be opened so that the first filter member may be replaced during the use of the thrombectomy device. This may allow for a more efficient and cleaner procedure in comparison to a system where the first filter member is not configured to be removed from the visualization device. In some embodiments, the filter device is operably connected to the pump by a first tube. In some embodiments, the filter device is operably connected to the reservoir by a second tube. In some embodiments, the visualization device is configured to allow a user to view a removed undesirable material during use of the thrombectomy device. Further, the visualization device is configured to be opened to enable removal of the captured undesirable material from the visualization device for assessing, testing, and imaging of the undesirable material.

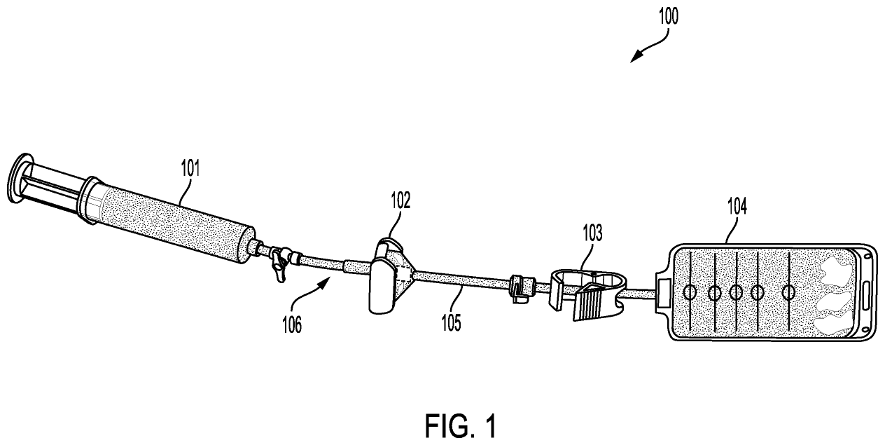

FIG. 1 illustrates a passive reinfusion system 100 with a syringe 101, a filter 102, and a reservoir 104 configured to be in fluid communication with one another. The passive reinfusion system 100 is configured to filter fluid that has already been aspirated from the body by using a thrombectomy device (not shown). The unfiltered fluid in the reservoir 104 may contain undesirable material that has been aspirated from the body. In some embodiments, the undesirable material has been aspirated substantially en bloc (wholly or partially) or has been fragmented during aspiration. The passive reinfusion system 100 further comprises a first tube 105 operably connected to the filter 102 and the reservoir 104 and configured to deliver fluid from the reservoir 104 to the filter 102. In some embodiments, the reservoir 104 can be disconnected from a thrombectomy device following aspiration of the undesirable material from the body and connected to the first tube 105. The passive reinfusion system 100 further comprises a second tube 106 operably connected to the filter 102 and the syringe 101 and configured to deliver fluid from the filter 102 to the syringe 101. In some embodiments, the syringe 101 is configured to draw fluid from the reservoir 104 as a user pulls back on a plunger of the syringe. In some embodiments, the filter 102 is configured to remove undesired material from the unfiltered fluid contained in the reservoir 104 as the unfiltered fluid passes through the filter 102. In some embodiments, the passive reinfusion system 100 further comprises a first fluid flow control device 103 configured to control or stop the flow of fluid through the first tube 105. In some embodiments, the first fluid flow control device 103 is one of a clamp a stopcock, or a valve. In some embodiments, the valve is a one-way check valve. In some embodiments, the passive reinfusion system 100 further comprises a second fluid flow control device 103 configured to control or stop the flow of fluid through the second tube 106. In some embodiments, the second fluid flow control device is one of a clamp a stopcock, or a valve. In some embodiments, the valve is a one-way check valve.

The filter 102 may be any filter known to one of skill in the art effective for filtering undesired material from fluid. In some embodiments, the filter 102 has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, or any value or range of values between any two of these values. In some embodiments, the filter 102 has a pore size of about 40 μm. In some embodiments, the filter 102 is configured to be removed from the passive reinfusion system 100 and replaced with a new filter 102. In some embodiments, the filter 102 is configured to filter fluid to a level acceptable for reinfusion into the patient. In some embodiments, the filter 102 includes a filter housing comprising a first filter membrane therein. In some embodiments, the first filter membrane is configured to be removed from filter housing and replaced with a second filter membrane.

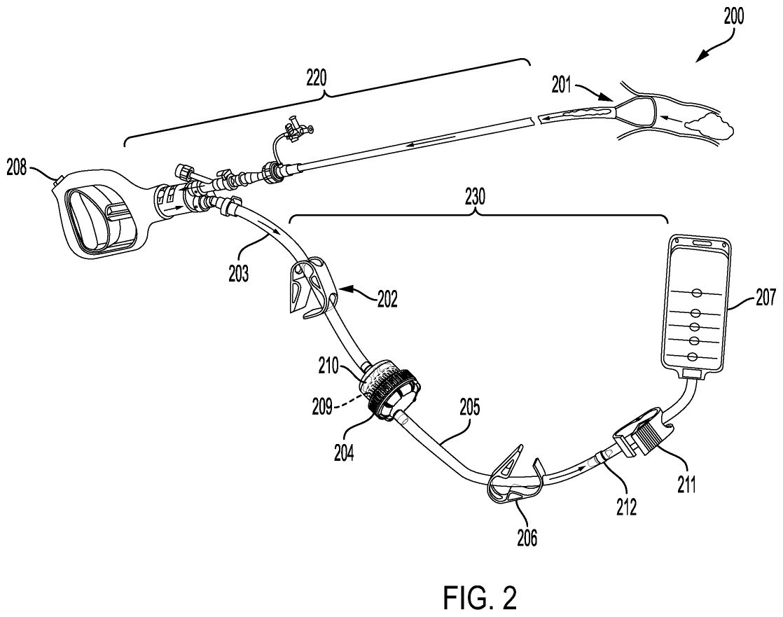

FIG. 2 illustrates a passive reinfusion system 200 having an aspiration system and/or thrombectomy device 220 and a filter system 230. The aspiration system 220 may comprise a suction cannula 201 and a pump 208 configured to aspirate fluid and/or undesirable material from a patient. In some embodiments, the pump 208 may comprise the aspiration device as described in U.S. patent application Ser. No. 17/170,752, or may comprise any manual mechanical aspiration source such as a syringe or hand pump, or source of aspiration such as a vacuum, peristaltic pump, centrifugal pump, or any other source of aspiration known in the art. In some embodiments, the pump 208 is configured to generate a suction force sufficient to aspirate fluid and undesirable material through the suction cannula 201 from the body. In some embodiments, the pump 208 is configured to generate a driving force through a passive reinfusion system 200.

The pump 208 may be operably connected to the filter system 230. The filter system 230 is configured to remove undesired material from the aspirated fluid. The filter system 230 comprises a first tube 203 operably connected to and in fluid communication with the pump 208 and a visualization device 204. The first tube 203 is configured to deliver unfiltered fluid from the pump 208 to the visualization device 204 such as, for example, upon application of a driving force from the pump 208. The visualization device 204 may comprise a first filter member 209 configured to filter undesired material from fluid as the fluid passes through the visualization device 204. In some embodiments, the visualization device 204 comprises a visualization chamber 210 configured to enable a user to visualize the captured undesirable material within the visualization device 204. The pump 208 is configured to create a driving force sufficient to force the unfiltered fluid to pass through the first filter member 209 of the visualization device 204. The filter system 230 may further comprise a second tube 205 operably connected to and in fluid communication with the visualization device 204 and a reservoir 207. The second tube 205 is configured to deliver fluid from the visualization device 204 to the reservoir 207 such as, for example, upon application of a driving force from the pump 208. In some embodiments, the second tube 205 is a single continuous tube fluidly connecting the visualization device 204 and reservoir 207. In another embodiment, the second tube 205 can include two tubes connected at connection 212, e.g., a quick connect, to enable removal of the reservoir 207 from the filter system 230. In some embodiments, the filter system 230 further comprises a first fluid flow control device 202 positioned about the first tube 203 and configured to control the flow of fluid through the first tube 203. In some embodiments, the filter system 230 further comprises a second fluid flow control device 206 positioned about the second tube 205 and configured to control the flow of fluid through the second tube 205. In some embodiments, the filter system 230 further comprises a third fluid flow control device 211 positioned about the second tube 205 and configured to control the flow of fluid through the second tube 205.

In some embodiments, each of the fluid flow control devices 202, 206, 211 is configured to control the flow of fluid within the filter system 230. Each of the fluid flow control devices 202, 206, 211 may be any fluid flow control device effective for sealing a tube. In some embodiments, each of the fluid flow control devices 202, 206, 211 is one of a clamp, a stopcock, or a valve. In some embodiments, the valve is a one-way check valve. In some embodiments, the first fluid flow control device 202 is configured to seal the first tube 203, upstream of the visualization device 204, and the second fluid flow control device 206 is configured to seal the second tube 205, downstream of the visualization device 204, to allow the visualization device 204 to be removed from the filter system 230. In some embodiments, the second fluid control device 206 and the third fluid flow control device 211 are configured to seal the second tube 205 to allow the reservoir 207 to be removed from the filter system 230. Sealing the tubes 203, 205 before removing and/or opening the visualization device 204 and removing the reservoir 207 may prevent a loss of fluid from the filter system 230. In some embodiments, each of the second fluid control device 206 and the third fluid control device 211 may be closed or sealed, and the reservoir 207 and third fluid control device 211 may be removed and/or disconnected from the filter system 230 at connection 212. In some embodiments, the reservoir 207 is removed from the filter system 230 and further filtered before being returned to the patient. In some embodiments, the reservoir 207 may be removed and coupled with first tube 105 of the embodiments described relative to FIG. 1 such that fluid within the reservoir 207 can be drawn into the syringe 101 through the filter 102 to capture any undesired material. In some embodiments, the reservoir 207 is configured to be replaced with a new reservoir (not shown) to allow for the continued aspiration and filtering of fluid from the body, while the filtered fluid of the removed reservoir 207 are being reinfused back to the body. The filtered fluid may be reinfused back to the body at any spaced location from the aspiration site, and in some embodiments may be reinfused back through the aspiration cannula in between suction or aspiration forces. Furthermore, the filtered fluid may be reinfused back to the body through any standard catheter or cannula known in the art. Alternatively, the filtered fluid may be stored to be reinfused at a later time; used for additional medical testing or biopsy; or is cleaned and/or prepared for blood transfusions to be used on another patient.

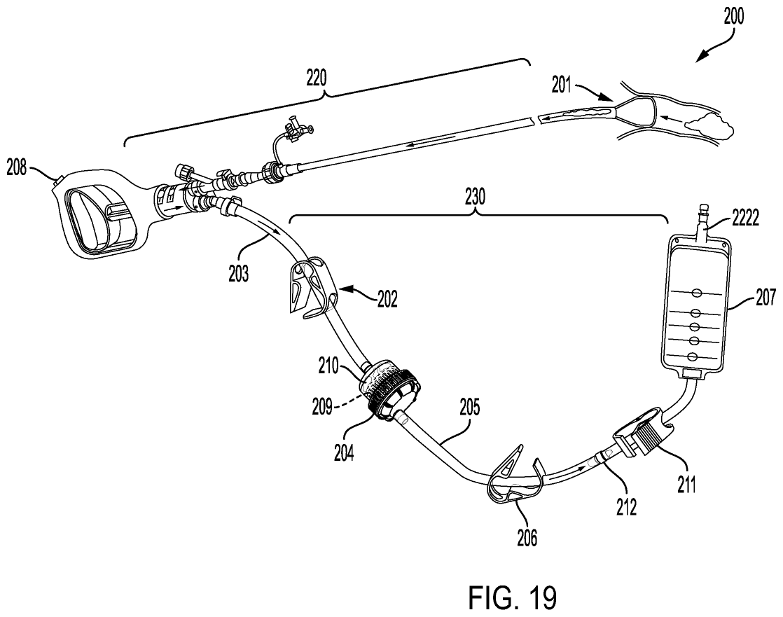

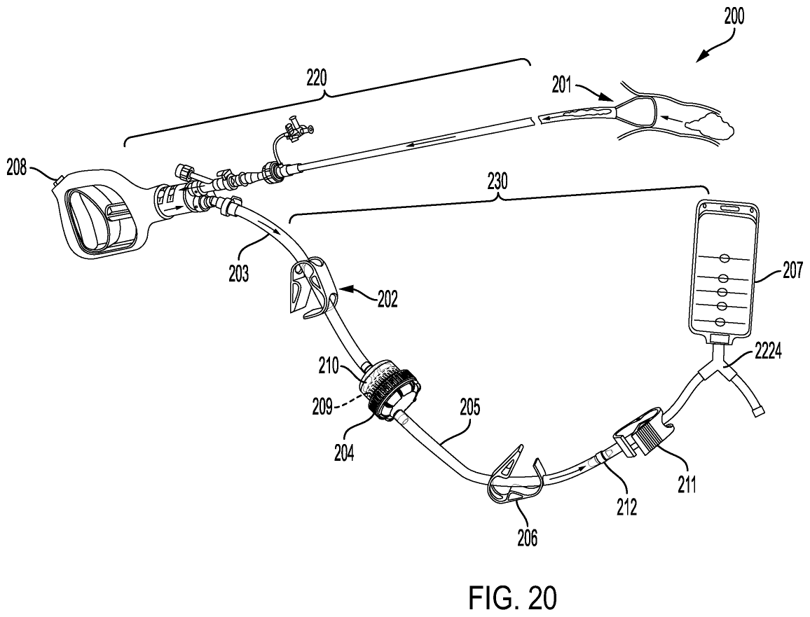

In some embodiments, as shown in FIG. 19 and FIG. 20, the reservoir 207 may further include a port 2222 positioned opposite of the tube 205 or a y-connector 2224 for coupling of the reservoir 207 to the first tube 105 of FIG. 1 in addition to the second tube 205. The port 2222 may be comprised of a standard luer connection, a valve (such as a one-way check valve), or another standard medical connection. These embodiments allow for fluid that is within the reservoir 207 to be drawn into the syringe 101 through the filter 102 to capture any undesired material while the reservoir 207 is still connected to the second tube 205. Such embodiments, allow for easier and more efficient passive reinfusion of filtered fluid back into the patient and avoid the need to disconnect the reservoir 207 from the second tube 205 which can be timely, cumbersome, and cause spillage. In addition, having a port 2222 or y-connector 2224 on the reservoir allows for de-airing of the reservoir 207 if needed. Further, having the ability to directly connect the passive reinfusion system 100 of FIG. 1 to the passive reinfusion system 200 of FIG. 2 allows the user to filter and reinfuse the fluid in the reservoir 207 at any time such as when the reservoir 207 is full or within a threshold time prior to the fluid in the reservoir 207 clotting (for example, if the fluid was resting too long within the reservoir 207.

In some embodiments, the visualization device 204 is configured to remove large bore thrombi from the fluid. In some embodiments, the visualization device 204 is transparent to allow for a user to view the contents of the visualization device 204. In some embodiments, the visualization device 204 is configured to open to allow for the removal of the first filter member 209 and the undesired material from the visualization device 204. In some embodiments, the first filter member 209 is configured to be removed from the visualization device 204 and replaced by a replacement filter member. The first filter member 209 may have any pore size effective for the removal of undesired material from the fluid while allowing the fluid to flow through the first filter member 209. In some embodiments, the first filter member 209 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member 209 has a pore size of about 750 μm to about 1000 μm. In some embodiments, the first filter member 209 comprises polycarbonate.

In some embodiments, the visualization device 204 is configured to be detached from the first tube 203 and the second tube 205 while the first and second fluid flow control devices 202, 206 are closed and/or sealed. In other embodiments, the visualization device 204 can be opened while the visualization device 204 is attached to tubes 203, 205 and fluid flow control devices 202, 206 are closed and/or sealed. The removal of the visualization device 204 from the filter system 230 allows for easier opening of the visualization device 204, easier replacement of the first filter member 209, and easier removal of the undesired material from the visualization device 204. Additionally, the removal of the visualization device 204 from the filter system 230 allows for imaging of the undesired material and allows the undesired material to be further tested. In some embodiments, the testing includes testing the removed undesired material to determine an infection and administer proper antibiotics. In some embodiments, the testing may include determining pathophysiology of the undesired material (e.g., whether the undesirable material contains fibrin, whether the undesirable material includes soft clot or hard clot, etc.) and/or an amount of undesired material removed from the patient. In some embodiments, the first fluid flow control device 202 is configured to seal the first tube 203 to prevent the flow of fluid from the pump 208 to the visualization device 204. This allows for the visualization device 204 to be removed from the filter system 230 while reducing the amount of fluid spilled from the filter system 230 and therefore increasing the amount of fluid returned to the patient in comparison to a system without the first fluid flow control device 202.

While it has been shown and described that the visualization device 204 is downstream of the pump 208, it is to be understood that aspects of the disclosure contemplate other arrangements of the pump 208 and the visualization device 204. For example, in another embodiment, the visualization device 204 may be positioned upstream of the pump 208 such that fluid is filtered as it is aspirated by the pump 208 prior to being discharged from the pump 208 into the reservoir 207.

The visualization device 204 provides numerous benefits including, but not limited to, a reduction in the amount of contrast used in identifying the thrombi in patient. For example, the clot visualization device 204 allows a user to visualize an amount of thrombi removed from the patient, thereby enabling the user to determine whether to proceed with aspiration and forego an additional round of imaging and contrast injection for the patient. During conventional thrombectomy procedures, physicians perform multiple rounds of imaging in order to assess how much thrombus remains in the vessel and whether to continue aspirating. There is a risk to the patient with each round of imaging, and too much contrast can cause renal function impairment. The ability to forgo even just one additional round of contrast injection benefits the patient. Further, the ability to open the visualization device 204 and remove captured undesired material enables the user to take pictures of the captured material in order to be shared with the patient and colleagues, and even post on their social media pages. Additionally, the visualization device 204 allows the user to visualize the removed undesirable material and compare with pre-procedure images.

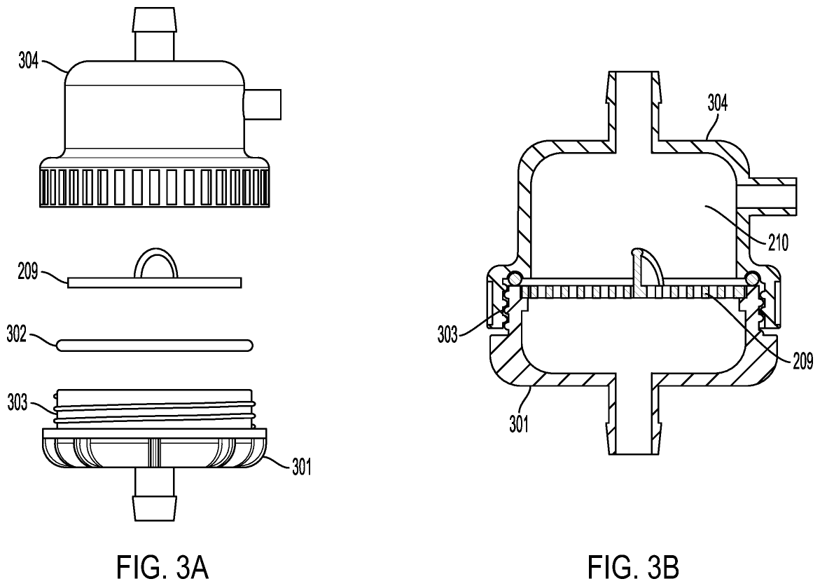

FIG. 3A illustrates an exploded view of the visualization device 204. In some embodiments, the visualization device 204 may comprise an upper housing 304, a lower housing 301, the first filter member 209 and an O-ring 302. The upper housing 304 and a lower housing 301 are configured to be releasably securely connected to each other. In some embodiments, the upper housing 304 may be configured to be threadably connected and/or mated with the lower housing 301. In some embodiments, the O-ring 302 is configured to seal the visualization device 204 and ensure that fluid does not leak out of the visualization device 204 at the locking mechanism 303.

FIG. 3B illustrates a cross-section of the visualization device 204. The visualization device 204 may comprise the first filter member 209 configured to filter fluid passing through the visualization device 204 and a locking mechanism 303 configured to allow the visualization device 204 to be opened and closed. In some embodiments, the locking mechanism 303 may be one of a screw cap, a pin slide twist lock, a snap lock, a sliding lock, a gate lock, or a visual indicator lock. The first filter member 209 is positioned substantially in the center of the visualization device 204 and may be configured to filter fluid as the fluid flows through the first filter member 209. In some embodiments, the visualization device 204 further comprises a visualization chamber 210 defined within the upper housing 304 and/or the lower housing 301 upstream of the filter membrane 209. The upper housing 304 and the lower housing 301 may be transparent and are configured to allow the user to visualize the filtered undesirable material in the visualization chamber 210 of the visualization device 204 after the fluid has been passed through the first filter member 209.

FIG. 3C illustrates an enlarged view of the visualization device 204 configured to remove undesired material from fluid. The visualization device 204 may comprise an inlet 305 configured to allow fluid to enter the visualization device 204 and an outlet 307 configured to allow fluid to exit the visualization device 204. In some embodiments, the visualization device 204 further comprises a flushing port 306 configured to allow a flushing liquid to be injected into the visualization device 204. In some embodiments, the flushing port 306 is a luer lock. Flushing the visualization device 204 may increase visibility within the visualization device 204 as fluid is forced out of the outlet 307. This may enable a user to visualize the undesired material present within the visualization device 204 more easily than in a system where the visualization device 204 is not flushed.

In some embodiments, the inlet 305 is configured to attach the visualization device 204 to a first tube, e.g., first tube 203 of FIG. 2. In some embodiments, the outlet 307 is configured to attach the visualization device 204 to a second tube, e.g., second tube 205 of FIG. 2. The inlet 305 and the outlet 307 may be any port effective for connecting to a tube. In some embodiments, each of the inlet 305 and the outlet 307 is a barb port or a quick connect. In some embodiments the visualization device 204 has an internal volume of about 30 cc, about 35 cc, about 40 cc, about 45 cc, about 50 cc, about 55 cc, about 60 cc, about 65 cc, about 70 cc, about 75 cc, about 80 cc, about 85 cc, about 90 cc, about 95 cc, about 100 cc, about 150 cc, about 200 cc, about 250 cc, about 300 cc, about 350 cc, about 400 cc, about 450 cc, about 500 cc, about 550 cc, about 600 cc, about 650 cc, about 700 cc, about 750 cc, about 800 cc, about 850 cc, about 900 cc, about 950 cc, about 1000 cc, or any value or range of values between any two of these values.

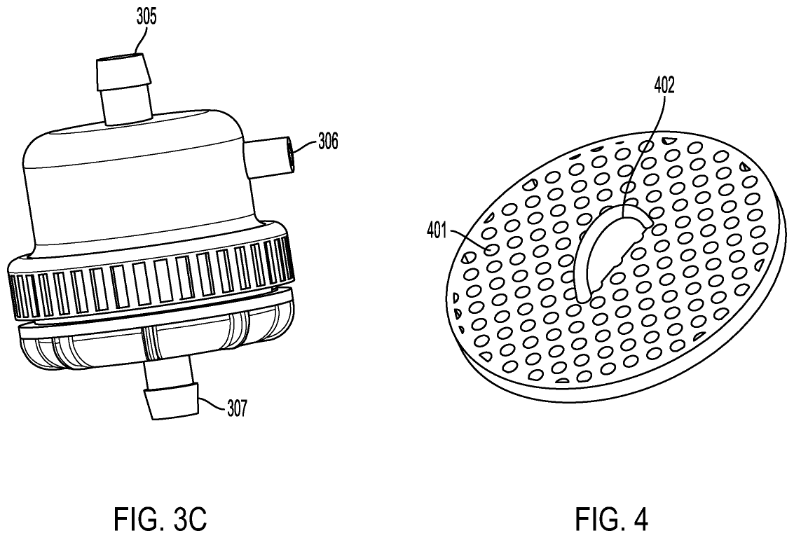

FIG. 4 illustrates an enlarged view of the first filter member 209 configured to filter fluid within a visualization device. In some embodiments, the first filter member 209 comprises a plurality of pores 401 configured to allow fluid to flow through the first filter member 209 and to prevent the flow of undesired material through the first filter member 209. The first filter member 209 may have any pore size effective for the removal of large bore thrombi from the fluid while allowing the fluid to flow through the first filter member 209. In some embodiments, the first filter member 209 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member 209 has a pore size of about 750 μm to about 1000 μm.

In some embodiments, the first filter member 209 comprises a removal mechanism 402 configured to allow the filter member to be removed from the visualization device 204. In some embodiments, the removal mechanism 402 is one of a tab or a handle. The first filter member 209 may be any shape effective for filtering undesirable material within a visualization device 204. In some embodiments, the first filter member 209 is shaped like a disk. In some embodiments, the surface of the first filter member 209 comprises a coating configured to reduce the adhesion of fluid and undesirable material to the first filter member 209 as compared to an uncoated filter member. In some embodiments, the coating is comprised of PTFE and/or any other now known or later developed anti-thrombus coating.

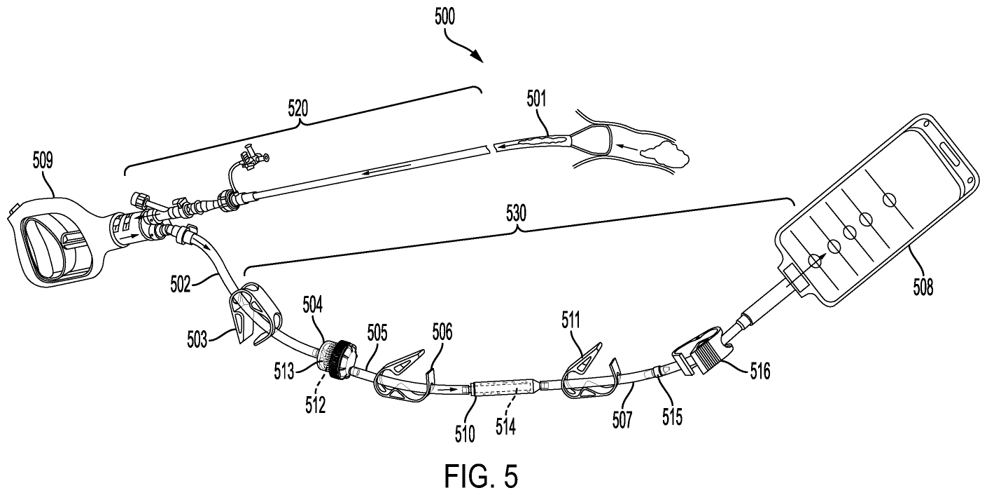

FIG. 5 shows another embodiment of the disclosure. This embodiment is similar to that of FIG. 2, however, this embodiment includes a filter housing in addition to a visualization tool. FIG. 5 illustrates a passive reinfusion system 500 comprising an aspiration system and/or thrombectomy device 520 and a filter system 530. The aspiration system 520 comprises a suction cannula 501 and a pump 509 configured to aspirate fluid and undesirable material from a patient. The pump 509 may comprise the aspiration device as described in U.S. patent application Ser. No. 17/170,752, or may comprise any manual mechanical aspiration source such as a syringe or hand pump, or source of aspiration such as a vacuum, peristaltic pump, centrifugal pump, or any other source of aspiration known in the art. In some embodiments, the pump 509 may be operably connected to and in fluid communication with a suction cannula 501 having a distal end placed in the body near the undesirable material. In some embodiments, the pump 509 is configured to generate a suction force sufficient to aspirate fluid and undesirable material through the suction cannula 501 from the body. In some embodiments, the pump 509 is configured to generate a driving force through the passive reinfusion system 500.

The pump 509 may be operably connected to the filter system 530. The filter system 530 is configured to remove undesired material from the aspirated fluid. The filter system 530 may comprise a first tube 502 operably connected to and in fluid communication with the pump 509 and a visualization device 504. The first tube 502 may be configured to deliver unfiltered fluid from the pump 509 to the visualization device 504 such as, for example, upon application of a driving force from the pump 509. The visualization device 504 may comprise a first filter member 512 configured to filter undesired material from fluid as the fluid passes through the visualization device 504. In some embodiments, the first filter member is a screen or a mesh. The visualization device 504 may also comprise a visualization chamber 513 configured to allow the user to visualize the undesirable material that has been filtered from the unfiltered fluid received from the pump. The filter system 530 may further comprise a second tube 505 operably connected to and in fluid communication with the visualization device 504 and a filter housing 510. The filter housing 510 comprises a second filter member 514 configured to deliver fluid from the visualization device 504 to the filter housing 510 such as, for example, upon application of a driving force from the pump 509. The second filter member 514 is configured to provide a second stage filtration of partially filtered fluid after the partially filtered fluid has passed through the first filter member 512 of the visualization device 504. In some embodiments, the filter system 530 comprises a third tube 507 configured to deliver the filtered fluid from the filter housing 510 to a reservoir 508 such as, for example, upon application of a driving force from the pump 509. In some embodiments, the third tube 507 is a single continuous tube fluidly connecting the filter housing 510 and reservoir 508. In another embodiment, the third tube 507 can include two tubes connected at connection, e.g., a quick connect, 515 to enable removal of the reservoir 508 from the filter system 530. In some embodiments, the filter system 530 further comprises a first fluid flow control device 503 positioned about the first tube 502 and configured to control the flow of fluid through the first tube 502. In some embodiments, the filter system 530 further comprises a second fluid flow control device 506 positioned about the second tube 505 and configured to control the flow of fluid through the second tube. In some embodiments, the filter system 530 comprises a third fluid control flow device 511 positioned about the third tube 507 and configured to control the flow of fluid through the third tube 507.

In some embodiments, the visualization device 504 comprises a first filter member 512 configured to filter fluid as the unfiltered fluid has passes through the visualization device 504. The first filter member 512 may be any filter effective for removing undesired material from fluid known to one of skill in the art. In some embodiments, the first filter member 512 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member 512 has a pore size of about 750 μm to about 1000 μm.

In some embodiments, the second filter member 514 is configured to filter fluid after the fluid has passed through the first filter member 512. In some embodiments the first filter member 512 comprises a larger pore size or a larger filtration membrane than the second filter member 514. In some embodiments, the second filter member 514 may be any filter effective for removing undesired material from fluid known to one of skill in the art. In some embodiments, the second filter member 514 has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member 514 has a pore size of about 40 μm. In some embodiments, the second filter member 514 is configured to filter fluid to a level acceptable for reinfusion into the patient. In some embodiments, the second filter member 514 is configured to be removed from the filter system 530. In some embodiments, the second filter member 514 is configured to be replaced after the second filter member 514 has filtered a threshold amount of undesired material. In some embodiments, the first filter member 512 is comprised of polycarbonate. In some embodiments, the second filter member 514 is comprised of one of polypropylene, polyester, or ePTFE.

In some embodiments, each of the first fluid flow control device 503, the second fluid flow control device 506 and the third fluid flow control device 511 is configured to control the flow of fluid within the filter system 530. Each of the first fluid flow control device 503, the second fluid flow control device 506 and the third fluid flow control device 511 may be any fluid flow control device effective for controlling the flow of fluid within a tube. In some embodiments, each of the first fluid flow control device 503, the second fluid flow control device 506 and the third fluid flow control device 511 is one of a clamp, a stopcock, or a valve. In some embodiments, the valve is a one-way check valve. In some embodiments, the first fluid flow control device 503 and the second fluid flow control device 506 are configured to allow for the sealing of the tubes 502 and 505 to allow for the removal of the visualization device 504 from the filter system 530. For example, the first fluid flow control device 503 is configured to seal or stop fluid flow through the first tube 502, upstream of the visualization device 504, and the second fluid flow control device 506 is configured to seal or stop fluid flow through the second tube 505, downstream of the visualization device 504, to allow the visualization device 504 to be removed from the filter system 530. The fluid flow control devices 503, 506 can be sealed or close fluid flow therethrough to allow for the removal of the visualization device 504 so the visualization device 504 can be emptied and/or the first filter member 512 can be replaced. In some embodiments, the second fluid flow control device 506 and the third fluid flow control device 511 are configured to allow for the sealing of tubes 505 and 507 to allow for removal of the second filter member 514 from the filter system 530. For example, the second fluid control device 506 is configured to seal the first tube 505, upstream of the filter housing 510, and the third fluid flow control device 511 is configured to seal the third tube 507, downstream of the filter housing 510, to allow the filter housing 510 to be removed from the filter system 530. More specifically, the fluid flow control devices 506, 511 can be closed to allow for the removal of the filter housing 510 so the filter housing 510 can be emptied and/or the second filter member 514 can be replaced. In some embodiments, the third fluid flow control device 511 and fourth fluid flow control device 516 are configured to allow for the sealing of the third tube 507 for the removal of the reservoir 508 from the filter system 530. In some embodiments, each of the third fluid control device 511 and the fourth fluid control device 516 may be closed or sealed and the reservoir 508 and fourth fluid control device 516 may be removed and/or disconnected from the filter system 530 at connection 515. In some embodiments, the reservoir 508 is configured to be removed from the filter system 530 and is used to reinfuse the filtered fluid into the patent. In some embodiments, the reservoir 508 is configured to be replaced in the filter system 530 with another reservoir such that the thrombectomy procedure can continue while fluid from reservoir 508 is reinfused within the patient.

While it has been shown and described that the visualization device 504 and filter housing 510 are downstream of the pump 509, it is to be understood that aspects of the disclosure complete other arrangements of the pump 509, the visualization device 504 and filter housing 510. For example, in another embodiment, both of the visualization device 504 and the filter housing 510 may be positioned upstream of the pump 509 such that fluid is filtered as it is aspirated by the pump 509 prior to being discharged into the reservoir 508. However, in another embodiment, the visualization device 504 may be positioned upstream of the pump 509 such that fluid is at least partially filtered as it is aspirated by the pump 509. In this embodiment, the filter housing 510 may be positioned downstream of the pump 509 so as to capture any smaller sized undesired material and/or debris not captured by the visualization device 504 prior to passing into the reservoir 508.

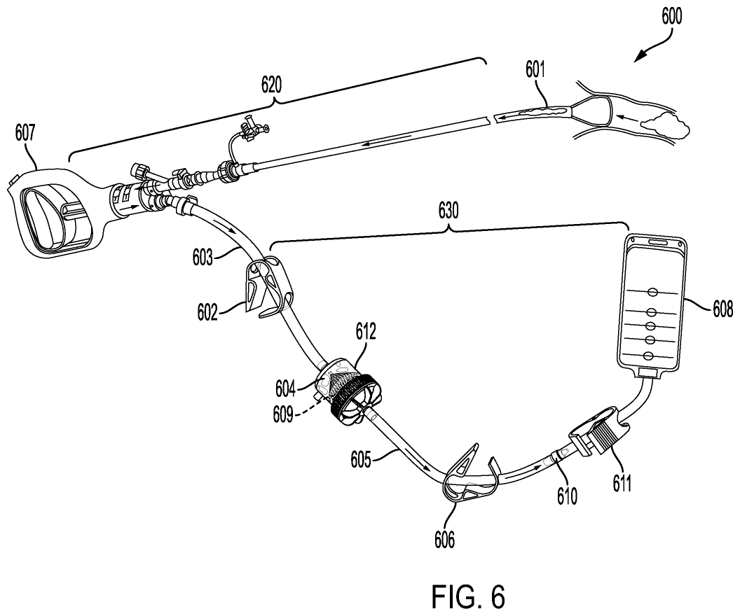

FIG. 6 illustrates another embodiment of the disclosure. This embodiment is similar to that of FIG. 5, however, the visualization device 604 according to this embodiment comprises both a first filter member 609 and a second filter member (not shown), such that an additional filter housing is not included. In some embodiments, the first filter member 609 and the second filter member have varying or different pore sizes or filter membrane sizes within the visualization device 604. FIG. 6 illustrates a passive reinfusion system 600 comprising an aspiration system and/or thrombectomy device 620 and a filter system 630. The aspiration system 620 comprises a pump 607 configured to be in fluid communication with a suction cannula 601 configured to be placed in the body and a distal end near the undesirable material. In some embodiments, the pump 607 is configured to generate a suction force sufficient to aspirate fluid and undesirable material through the suction cannula 601 from the body. In some embodiments, the pump 607 is configured to generate a driving force through the passive reinfusion system 600. The pump 607 may comprise the aspiration device as described in U.S. patent application Ser. No. 17/170,752, or may comprise any manual mechanical aspiration source such as a syringe or hand pump, or source of aspiration such as a vacuum, peristaltic pump, centrifugal pump, or any other source of aspiration known in the art. In some embodiments, the passive reinfusion system 600 is configured for the pump 607 to create a suction force or aspiration force sufficient to aspirate undesired material and fluid from the body through a suction cannula 601. In some embodiments, the pump 607 is configured to generate a driving force through the passive reinfusion system 600.

The pump 607 may be operably connected to the filter system 630. The filter system 630 is configured to remove undesired material from the aspirated fluid. The filter system 630 may comprise a first tube 603 operably connected to and in fluid communication to the pump 607 and a visualization device 604. The first tube 603 is configured to deliver unfiltered fluid from the pump 607 to the visualization device 604 such as, for example, upon application of a driving force from the pump 607. The visualization device 604 may comprise a first filter member 609 and a second filter member, both configured to substantially filter the undesired material from fluid as the fluid passes through the visualization device 604. In some embodiments, the visualization device 604 comprises a visualization chamber 612 configured to allow the user to visualize the undesirable material that has been filtered from the unfiltered fluid. The pump 607 is configured to create a driving force sufficient to force the unfiltered fluid to pass through the first filter member 609 and second filter member. The filter system 630 may further comprise a second tube 605 operably connected and in fluid communication to the visualization device 604 and a reservoir 608. The second tube 605 is configured to deliver fluid from the visualization device 604 to the reservoir 608 such as, for example, upon application of a driving force from the pump 607. In some embodiments, the second tube 605 is a single continuous tube fluidly connecting the visualization device 604 and reservoir 608. In another embodiment, the second tube 605 can include two tubes connected at connection, e.g., a quick connect, 610 to enable removal of the reservoir 608 from the filter system 630. In some embodiments, the filter system 630 further comprises a first fluid flow control device 602 positioned about the first tube 603 and configured to control the fluid through the first tube 603. In some embodiments, the filter system 630 further comprises a second fluid flow control device 606 positioned about the second tube 605 and configured to control the flow of fluid through the second tube 605.

In some embodiments, the visualization device 604 comprises a first filter member 609 configured to filter fluid as the unfiltered fluid has passes through the visualization device 604. The first filter member 609 may be any filter effective for removing undesired material from fluid known to one of skill in the art. In some embodiments, the first filter member 609 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member 609 has a pore size of about 750 μm to about 1000 μm.

In some embodiments, the visualization device 604 comprises a second filter member configured to filter fluid as the unfiltered fluid has passes through the visualization device 604. In some embodiments the first filter member 609 comprises a larger pore size or a larger filtration membrane than the second filter member. The second filter member may be any filter effective for removing undesired material from fluid known to one of skill in the art. In some embodiments, the second filter member has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member has a pore size of about 40 μm. In some embodiments, the second filter member is configured to filter fluid to a level acceptable for reinfusion into the patient. In some embodiments, the second filter member is configured to be removed from the filter system 630 when the visualization device 604 is opened. In some embodiments, the second filter member is configured to be replaced after the second filter member has filtered a threshold amount of undesired material. The positioning of the second filter member within the visualization device 604 may enable easier use of the passive reinfusion system 600 in comparison to a system comprising a filter member positioned outside the visualization device 604.

In some embodiments, each of the first fluid flow control device 602, the second fluid flow control device 606, and the third fluid flow control device 611 is configured to control the flow of fluid within the filter system 630. Each of the first fluid flow control device 602, the second fluid flow control device 606, and the third fluid flow control device 611 can be any fluid flow control device effective for controlling the flow of fluid through a tube. In some embodiments, each of the first fluid flow control device 602, the second fluid flow control device 606, and the third fluid flow control device 611 is one of a clamp, a stopcock, or a valve. In some embodiments, the valve is a one-way check valve. In some embodiments, the first and second fluid flow control devices 602, 606 can be sealed to allow for removal of the visualization device 604. More specifically, the first fluid flow control device 602 is configured to seal the first tube 603, upstream of the visualization device 604, and the second fluid flow control device 606 is configured to seal the second tube 605, downstream of the visualization device 604, to allow the visualization device 204 to be removed from the filter system 630. In some embodiments, the second fluid control device 606 and the third fluid flow control device 611 are configured to seal the second tube 605 to allow the reservoir 608 to be removed from the filter system 630. Sealing the second tube 605 before removing the reservoir 608 may prevent a loss of fluid from the filter system 630 when the reservoir 608 is removed. In some embodiments, each of the second fluid control device 606 and the third fluid control device 611 may be closed or sealed and the reservoir 608 and third fluid control device 611 may be removed and/or disconnected from the filter system 630 at connection 610. In some embodiments, the reservoir 608 is configured to be removed from the filter system 630 and is used to reinfuse the filtered fluid into the patient. In some embodiments, the reservoir 608 is configured to be replaced in the filter system 630 with another reservoir such that the thrombectomy procedure can continue while fluid from reservoir 608 is reinfused within the patient.

While it has been shown and described that the visualization device 604 is downstream of the pump 607, it is to be understood that aspects of the disclosure contemplate other arrangements of the pump 607 and the visualization device 604. For example, in another embodiment, the visualization device 604 may be positioned upstream of the pump 607 such that fluid is filtered as it is aspirated by the pump 607 prior to being discharged from the pump 607 into the reservoir 608.

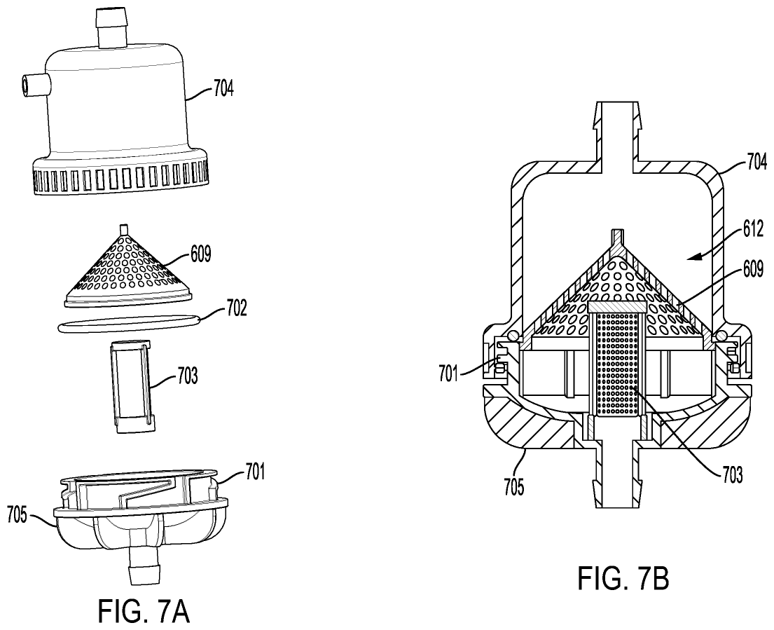

FIG. 7A illustrates an exploded view of the visualization device 604. In some embodiments, the visualization device 604 may comprise an upper housing 704, a lower housing 705, a first filter member 609, a second filter member 703, and an O-ring 702. The upper housing 704 and a lower housing 705 are configured to be releasably securely connected to each other. In some embodiments, the upper housing 704 may be configured to be threadably connected and/or mated with the lower housing 705. In some embodiments, the O-ring 702 is configured to seal the visualization device 604 and ensure that fluid does not leak out of the visualization device 604 at a locking mechanism 701.

FIG. 7B illustrates a cross-section of the visualization device 604. The visualization device 604 may comprise a first filter member 609 and a second filter member 703. In some embodiments, the visualization device 604 comprises a visualization chamber 612 defined within the upper housing 704 and/or the lower housing 705 upstream of the filter members 609, 703. The upper housing 704 and the lower housing 705 may be transparent and are configured to allow the user to visualize the captured undesirable material in the visualization chamber 612 of the visualization device 604 after the fluid has been passed through the filter members 609, 703. The visualization device 604 comprises a locking mechanism 701 configured to allow the visualization device 604 to be opened and closed, and an O-ring 702 configured to seal the locking mechanism 701. In some embodiments, the locking mechanism 701 is one of a screw cap, a pin slide twist lock, a snap lock, a sliding lock, a gate lock, threaded connection, or a visual indicator lock. The first filter member 609 may be positioned substantially in the center of the visualization device 604 and is configured to filter fluid as the fluid flows or is driven by the driving force from the pump 607 through the first filter member 609. The second filter member 703 may be positioned substantially in the center of the visualization device 604 and is configured to filter fluid as the fluid flows or is driven by the driving force from the pump 607 through the second filter member 703.

The first filter member 609 may be any filter effective for removing undesired material from fluid known to one of skill in the art. In some embodiments, the first filter member 609 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member 609 has a pore size of about 750 μm to about 1000 μm.

The second filter member 703 may be any filter effective for removing undesired material from fluid known to one of skill in the art. In some embodiments, the second filter member 703 has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member 703 has a pore size of about 40 μm. In some embodiments, the second filter member 703 is configured to filter fluid to a level acceptable for reinfusion into the patient. In some embodiments, the second filter member 703 is configured to be removed from the filter system 630 when the visualization device 604 is opened. In some embodiments, the second filter member 703 is configured to be replaced after the second filter member 703 has filtered a threshold amount of undesired material.

FIG. 7C illustrates an enlarged view of a visualization device 604 configured to remove undesired material from fluid. The visualization device 604 may comprise an inlet 706 configured to allow fluid to enter the visualization device 604 and an outlet 708 configured to allow fluid to exit the visualization device 604. In some embodiments, the visualization device 604 further comprises a flushing port 707 configured to allow a flushing liquid to be injected into the visualization device 604. In some embodiments, the flushing port 707 is a luer lock. Flushing the visualization device 604 may increase visibility within the visualization device 604 as fluid is forced out of the outlet 708. This may enable a user to visualize the undesired material present within the visualization device 604 more easily than in a system where the visualization device 604 is not flushed.

In some embodiments, the inlet 706 is configured to attach the visualization device 604 to a first tube, e.g., first tube 603 of FIG. 6. In some embodiments, the outlet 708 is configured to attach the visualization device to a second tube, e.g., second tube 605 of FIG. 6. The inlet 706 and the outlet 708 may be any port effective for connecting to a tube. In some embodiments, each of the inlet 706 and the outlet 708 is a barb port or a quick connect. In some embodiments the visualization device 604 has an internal volume of about 30 cc, about 35 cc, about 40 cc, about 45 cc, about 50 cc, about 55 cc, about 60 cc, about 65 cc, about 70 cc, about 75 cc, about 80 cc, about 85 cc, about 90 cc, about 95 cc, about 100 cc, about 150 cc, about 200 cc, about 250 cc, about 300 cc, about 350 cc, about 400 cc, about 450 cc, about 500 cc, about 550 cc, about 600 cc, about 650 cc, about 700 cc, about 750 cc, about 800 cc, about 850 cc, about 900 cc, about 950 cc, about 1000 cc, or any value or range of values between any two of these values.

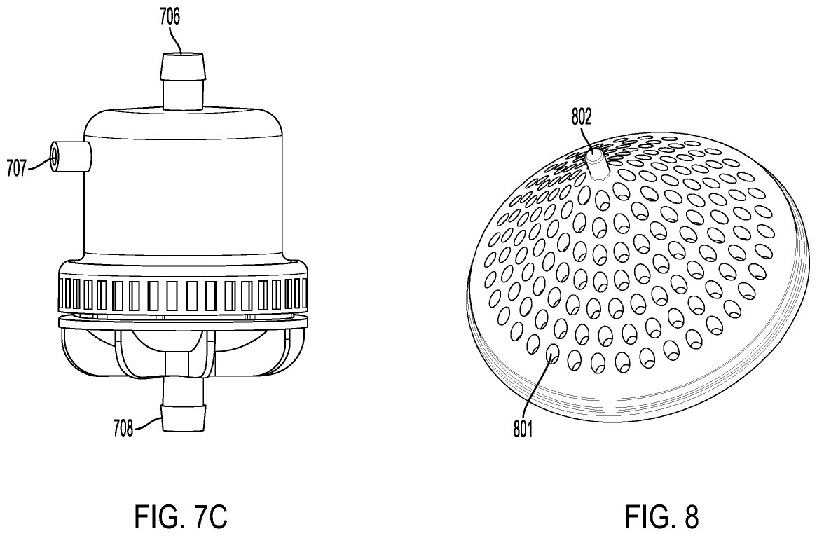

FIG. 8 illustrates one embodiment of the first filter member 609 and is configured to filter fluid within a visualization device. In some embodiments, the first filter member 609 comprises a plurality of pores 801 configured to allow fluid to flow through the first filter member 609 and to prevent the flow of large bore thrombi or other large undesirable material through the first filter member 609. The first filter member 609 may have any pore size effective for the removal of large bore thrombi or large undesirable material from the fluid while allowing the fluid to flow through the first filter member 609. In some embodiments, the first filter member 609 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member 609 has a pore size of about 750 μm to about 1000 μm.

In some embodiments, the first filter member 609 comprises a removal mechanism 802 configured to allow the first filter member 609 to be removed from a visualization device. In some embodiments, the removal mechanism 802 is one of a tab or a handle. The first filter member 609 may be any shape effective for filtering thrombi or undesirable material within a visualization device. In some embodiments, the first filter member 609 has a conical shape or a funnel shape. The use of a first filter member 609 with a conical shape may reduce the chance of the first filter member 609 clogging as compared to a disk-shaped filter member and provides additional room in the visualization device for the second filter member 703. In some embodiments, the surface of the first filter member 609 comprises a coating configured to reduce the adhesion of fluid to the first filter member 609 as compared to an uncoated filter member. In some embodiments, the coating is comprised of PTFE and/or any other now known or later developed anti-thrombus coating.

Each of the parts of the above systems may be operably connected to the tubing by any method effective for transporting fluid known to one of skill in the art. In some embodiments, each of the parts of the above systems is connected to the tubing by one of a barb port or a quick connect.

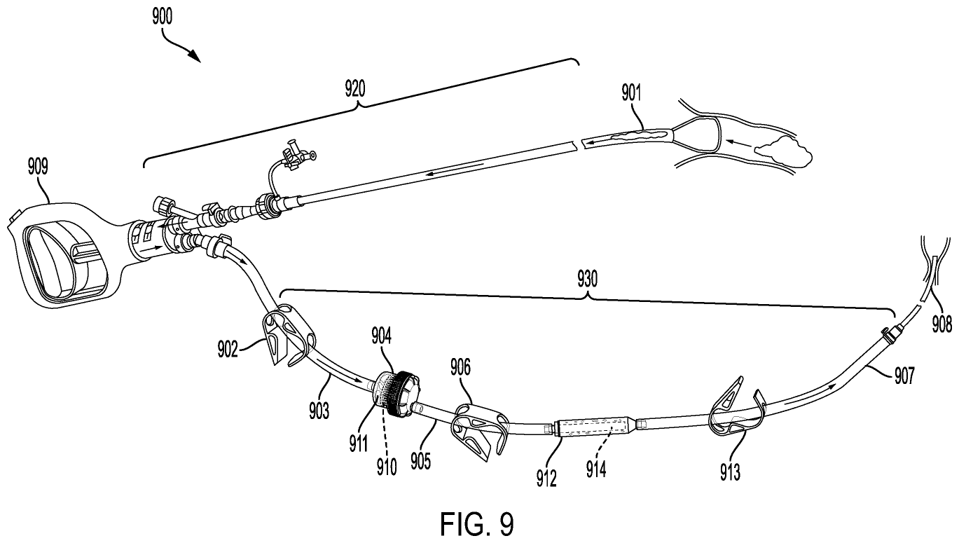

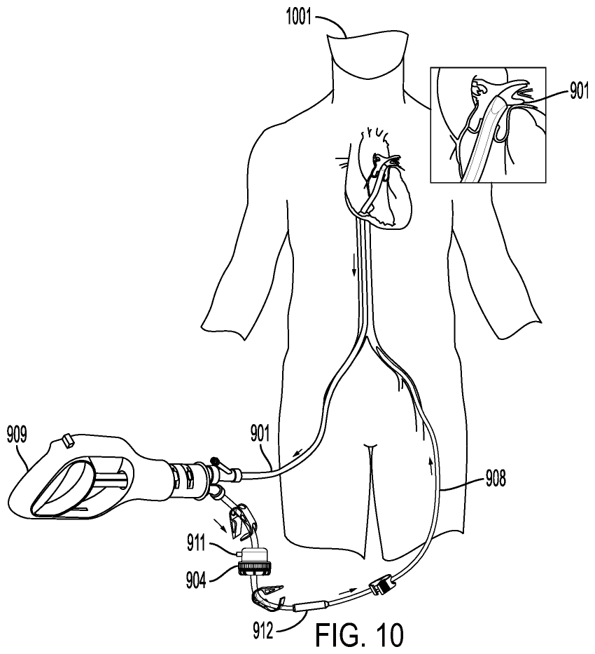

FIGS. 9 and 10 illustrate an active reinfusion system 900 with FIG. 10 showing the active reinfusion system 900 being used in a thrombectomy procedure. The active reinfusion system 900 comprises an aspiration system and/or thrombectomy device 920 and a filter system 930. The aspiration system 920 comprises a pump 909 in fluid communication with a suction and/or aspiration cannula 901 and is configured to generate an aspiration or suction force to aspirate unfiltered fluid comprising undesirable material from a patient 1001. The pump 909 may comprise the aspiration device as described in U.S. patent application Ser. No. 17/170,752, or may comprise any manual mechanical aspiration source such as a syringe or hand pump, or source of aspiration such as a vacuum, peristaltic pump, centrifugal pump, or any other source of aspiration known in the art. In some embodiments, the pump 909 is configured to generate a driving force to push the unfiltered fluid through the active reinfusion system 900 and directly reinfuse filtered fluid into the body. The active reinfusion system 900 may also comprise a first tube 903, a visualization device 904 comprising a first filter member 910 and a visualization chamber 911, a second tube 905, a filter housing 912 comprising a second filter member 914, a third tube 907, a reinfusion cannula 908, a first fluid flow control device 902, a second fluid flow control device 906, and a third fluid flow control device 913. In some embodiments, the first tube 903 may be operably connected to and in fluid communication with the pump 909 and the visualization device 904. The first tube 903 is configured to allow fluid to flow from the pump 909 to the visualization device 904 such as, for example, upon application of a driving force from the pump 909. The visualization device 904 may comprise a first filter member 910 configured to filter undesired material from fluid as the fluid passes into or is forced by the driving force through the visualization device 904. In some embodiments, the second tube 905 may be operably connected to and in fluid communication with the visualization device 904 and the filter housing 912 comprising the second filter member 914. The second filter member 914 is configured to provide a second stage filtration of the partially filtered fluid to filter additional undesired material from the fluid that was not previously filtered by the first filter member 910. The second tube 905 is configured to allow partially filtered fluid to flow from the visualization device 904 to the filter housing 912 such as, for example, upon application of a driving force from the pump 909. In some embodiments, the third tube 907 may be operably connected to and in fluid communication with the filter housing 912 and the reinfusion cannula 908. The third tube 907 is configured to allow filtered fluid to flow from the filter housing 912 to the reinfusion cannula 908 to be reinfused back to the body such as, for example, upon application of a driving force from the pump 909. In some embodiments, the reinfusion cannula 908 has a size of about 14 Fr, about 15 Fr, about 16 Fr, about 17 Fr, about 18 Fr, about 19 Fr, about 20 Fr, about 21 Fr, about 22 Fr, or any value or range of values between any two of these values.

The reinfusion cannula 908 is configured to be placed in the body in a spaced relationship with the aspiration cannula 901. In some embodiments, the first fluid flow control device 902 may be positioned about the first tube 903 and is configured to control the flow of fluid within the first tube 903. In some embodiments, the second fluid flow control device 906 may be positioned about the second tube 905 and is configured to control the flow of fluid within the second tube 905. In some embodiments, the third fluid flow control device 913 may be positioned about the third tube 907 and is configured to control the flow of the fluid within the third tube 907.

In some embodiments, the visualization device 904 comprises a first filter member 910 configured to filter fluid as the unfiltered fluid passes through the visualization device 904. The first filter member 910 may be any filter effective for removing undesired material from fluid known to one of skill in the art. In some embodiments, the first filter member 910 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1100 μm, about 1200 μm, about 1250 μm, about 1300 μm, about 1350 μm, about 1400 μm, about 1450 μm, about 1500 μm, or any value or range of values between any two of these values. In some embodiments, the first filter member 910 has a pore size of about 750 μm to about 1000 μm. In some embodiments, the first filter member 910 is configured to be removed from the filter system 930. In some embodiments, the first filter member 910 is configured to be replaced after the first filter member 910 has filtered a threshold amount of undesired material.

In some embodiments, the second filter member 914 is configured to provide a second stage filtration of partially filtered fluid after the partially filtered fluid has passed through the first filter member 910 of the visualization device 904. The second filter member 914 may be any filter effective for removing undesired material from fluid known to one of skill in the art. In some embodiments, the second filter member 914 has a pore size of about 5 μm, about 10 μm, about 10 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member 914 has a pore size of about 40 μm. In some embodiments, the second filter member 914 is configured to filter fluid to a level acceptable for reinfusion into the patient 1001. The second filter member 914 can also be configured to remove any debris within the filter system 930 that are not captured by the first filter member 910. In some embodiments, the second filter member 914 is configured to be removed from the filter system 930. In some embodiments, the second filter member 914 is configured to be replaced after the second filter member 914 has filtered a threshold amount of undesired material.

In some embodiments, each of the first fluid flow control device 902, the second fluid flow control device 906, and the third fluid flow control device 913 are configured to control the flow of fluid within the filter system 930. Each of the first fluid flow control device 902, the second fluid flow control device 906, and the third fluid flow control device 913 may be any fluid flow control device effective for control the flow of fluid within a tube. In some embodiments, each of the first fluid flow control device 902, the second fluid flow control device 906 and the third fluid flow control device 913 is one of a clamp or a stopcock. In some embodiments, the first and second fluid flow control devices 902, 906 can be sealed and fluid flow therethrough is stopped to allow for removal of the visualization device 904. More specifically, fluid flow control devices 902, 906 can be closed to allow for removal of the visualization device 904 to enable the visualization device to be emptied and one or more of the filter members therein to be replaced. In some embodiments, the second and third fluid flow control devices 906, 913 can be sealed to allow for removal of the filter housing 912. In some embodiments, the third fluid flow control device 913 is configured to allow for the sealing of the third tube 907 to prevent the flow of fluid to the reinfusion cannula 908.

In some embodiments, the active reinfusion system 900 comprises a port (not shown) configured for either priming the active reinfusion system 900, such as for de-airing the active reinfusion system 900, or for introducing a secondary device co-axially through the reinfusion cannula or the injection of a fluid into the active reinfusion system 900. In some embodiments, the fluid injected into the active reinfusion system 900 is one of saline, a drug, contrast, anti-thrombogenic, or other fluids. The port may be positioned adjacent to the reinfusion cannula 908. The port may be configured to prime the active reinfusion system 900 and allow for the removal of air from the active reinfusion system 900 prior to use of the active reinfusion system 900 to ensure that air is not introduced into the patient 1001. In some embodiments, the port is one of a valve, stopcock, or a leur lock. In some embodiments, the valve is a one-way check valve. In some embodiments, the active reinfusion system 900 further comprises an inlet port configured to allow liquid to be introduced into the active reinfusion system 900 and to force air out of the active reinfusion system 900 prior to use of the active reinfusion system 900. In some embodiments, the inlet port is a luer lock. The inlet port may be positioned at any position on the active reinfusion system 900 effective to introduce fluid to the active reinfusion system 900 and remove air from the active reinfusion system 900 including, but not limited to, the first fluid flow control device 902, the first tube 903, the visualization device 904, the second tube 905, the second fluid flow control device 906, the third fluid flow control device 913, or the third tube 907.

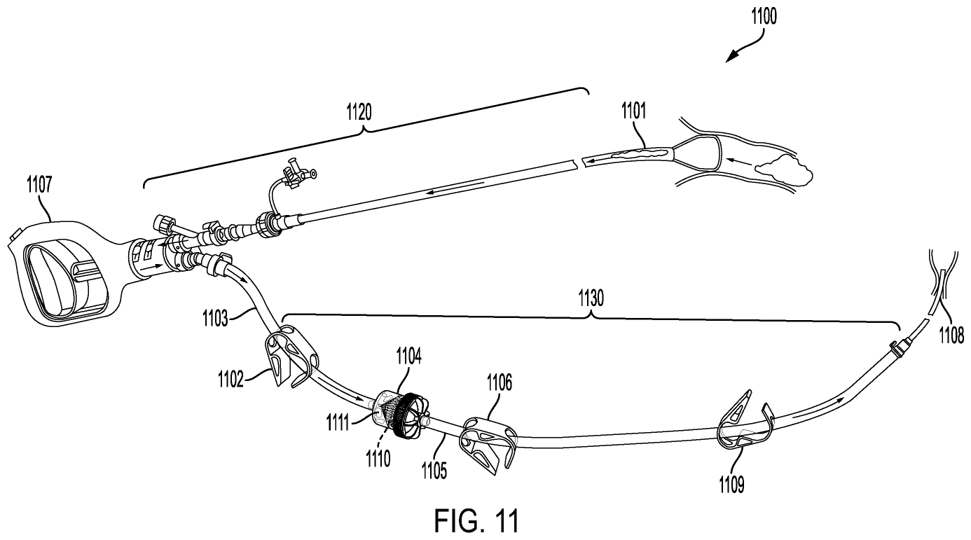

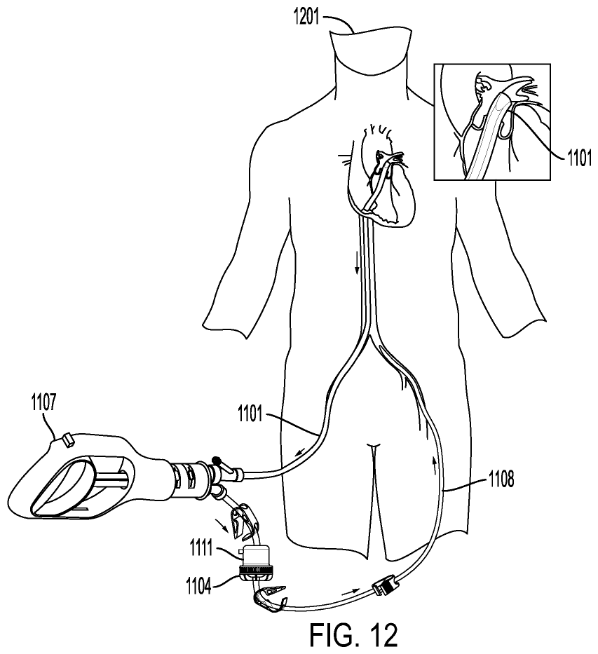

FIGS. 11-12 illustrate an active reinfusion system 1100 similar to that of FIGS. 9-10, however, the visualization device 1104 of FIGS. 11-12 comprises both a first filter member 1110 and a second filter member (not shown). In some embodiments, the first filter member 1110 and the second filter member have varying or different port sizes or filter membrane sizes within the visualization device 1104. The active reinfusion system 1100 comprises an aspiration system and/or thrombectomy device 1120 and a filter system 1130. The aspiration system 1120 comprises a pump 1107 in fluid communication with a suction and/or aspiration cannula 1101 and configured to generate an aspiration or suction force to aspirate unfiltered fluid comprising undesirable material from a patient 1201. The pump 1107 may comprise the aspiration device as described in U.S. patent application Ser. No. 17/170,752, or may comprise any manual mechanical aspiration source such as a syringe or hand pump, or source of aspiration such as a vacuum, peristaltic pump, centrifugal pump, or any other source of aspiration known in the art. In some embodiments, the pump 1107 is configured to generate a driving force to push the unfiltered fluid through the active reinfusion system 1100 directly reinfuse filtered fluid into the body. The active reinfusion system 1100 may also comprise a first tube 1103, a visualization device 1104 comprising a first filter member 1110 and a second filter member, a second tube 1105, a reinfusion cannula 1108, a first fluid flow control device 1102, a second fluid flow control device 1106, and a third fluid flow control device 1109.

In some embodiments, the first tube 1103 may be operably connected to and in fluid communication with the pump 1107 and the visualization device 1104 and configured to allow fluid to flow from the pump 1107 to the visualization device 1104. In some embodiments, the visualization device 1104 may comprise a first filter member 1110 configured to filter undesired material from fluid as the fluid passes or is forced by the driving force through the visualization device 1104. In some embodiments, the visualization device 1104 may comprise a second filter member configured to filter undesired material from fluid as the fluid passes or is forced by the driving force through the visualization device 1104. The visualization device 1104 may also comprise a visualization chamber 1111 configured to allow the user to visualize and see the filtered undesirable material. In some embodiments, the second tube 1105 may be operably connected to and in fluid communication with the visualization device 1104 and the reinfusion cannula 1108 and is configured to allow filtered fluid to flow from the visualization device 1104 to the reinfusion cannula 1108 to be reinfused back to the body. The reinfusion cannula 1108 may be any size effecting for reinfusing the filtered fluid into the patient 1201. In some embodiments, the reinfusion cannula 1108 has a size of about 14 Fr, about 15 Fr, about 16 Fr, about 17 Fr, about 18 Fr, about 19 Fr, about 20 Fr, about 21 Fr, about 22 Fr, or any value or range of values between any two of these values.

The reinfusion cannula 1108 is configured to be placed in the body in a spaced relationship with the aspiration cannula 1101. In some embodiments, the first fluid flow control device 1102 may be configured to control the flow of fluid within the first tube 1103 about a length thereof. In some embodiments, the second fluid flow control device 1106 may be configured to control the flow of fluid within the second tube 1105 about a length thereof.

In some embodiments, the visualization device 1104 comprises a first filter member 1110 configured to filter fluid as the unfiltered fluid has passes through the visualization device 1104. The first filter member 1110 may be any filter effective for removing undesired material from fluid known to one of skill in the art. In some embodiments, the first filter member 1110 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1100 μm, about 1200 μm, about 1250 μm, about 1300 μm, about 1350 μm, about 1400 μm, about 1450 μm, about 1400 μm, or any value or range of values between any two of these values. In some embodiments, the first filter member 1110 has a pore size of about 750 μm to about 1000 μm. In some embodiments, the first filter member 1110 is configured to be removed from the filter system 1130. In some embodiments, the first filter member 1110 is configured to be replaced after the first filter member 1110 has filtered a threshold amount of undesired material.

In some embodiments, the second filter member is configured to provide a second stage filtration of partially filtered fluid after the partially filtered fluid has passed through the first filter member 1110 of the visualization device 1104. The second filter member may be any filter effective for removing undesired material from fluid known to one of skill in the art. In some embodiments, the second filter member has a pore size of about 5 μm, about 10 μm, about 10 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member has a pore size of about 40 μm. In some embodiments, the second filter member is configured to filter fluid to a level acceptable for reinfusion into the patient. In some embodiments, the second filter member is configured to be removed from the filter system 1130. In some embodiments, the second filter member is configured to be replaced after the second filter member has filtered a threshold amount of undesired material.

In some embodiments, each of the first fluid flow control device 1102, the second fluid flow control device 1106, and the third fluid flow control device 1109 are configured to control the flow of fluid within the filter system 1130. Each of the first fluid flow control device 1102, the second fluid flow control device 1106, and the third fluid flow control device 1109 may be any fluid flow control device effective for control the flow of fluid within a tube. In some embodiments, each of the first fluid flow control device 1102, the second fluid flow control device 1106 and the third fluid flow control device 1109 is one of a clamp or a stopcock. In some embodiments, the first and second fluid flow control devices 1102, 1106 can be sealed and fluid flow therethrough is stopped to allow for removal of the visualization device 1104. More specifically, fluid flow control devices 1102, 1106 can be closed to allow for removal of the visualization device 1104 to enable the visualization device to be emptied and one or more of the filter members therein to be replaced. In some embodiments, the third fluid flow control device 1109 is configured to allow for the sealing of the second tube 1105 to prevent the flow of fluid to the reinfusion cannula 1108.

In some embodiments, the active reinfusion system 1100 comprises a port (not shown) configured for either priming the active reinfusion system 1100, such as for de-airing the active reinfusion system 1100, or for introducing a secondary device co-axially through the reinfusion cannula or the injection of a fluid into the active reinfusion system 1100. In some embodiments, the fluid injected into the active reinfusion system 1100 is one of saline, a drug, contrast, anti-thrombogenic, or other fluids. The port may be positioned adjacent to the reinfusion cannula 1108. The port may be configured to prime the active reinfusion system 1100 and allow for the removal of air from the active reinfusion system 1100 prior to use of the active reinfusion system 1100 to ensure that air is not introduced into the patient 1201. In some embodiments, the port is one of a valve, stopcock, or a leur lock. In some embodiments, the valve is a one-way check valve. In some embodiments, the active reinfusion system 1100 further comprises an inlet port configured to allow liquid to be introduced into the active reinfusion system 1100 and to force air out of the active reinfusion system 1100 prior to use of the active reinfusion system 1100. In some embodiments, the inlet port is a luer lock. The inlet port may be positioned at any position on the active reinfusion system 1100 effective to introduce fluid to the active reinfusion system 1100 and remove air from the active reinfusion system 1100 including, but not limited to, the first fluid flow control device 1102, the first tube 1103, the visualization device 1104, the second tube 1105, the second fluid flow control device 1106, or the third fluid flow control device 1109.

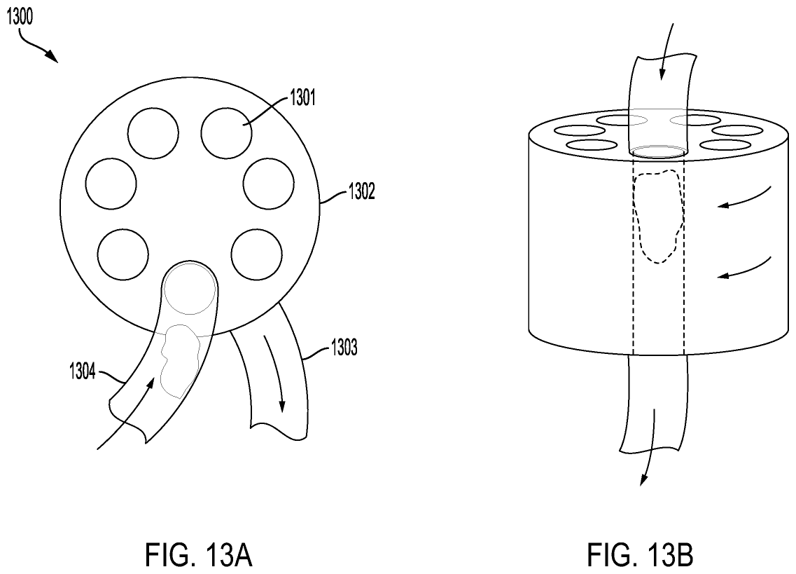

FIG. 13A-13B illustrate a revolver filter device 1300 configured to filter undesired material from unfiltered fluid that has been aspirated from the body. The revolver filter device 1300 may comprise a rotating device 1302 having a plurality of filter chambers 1301. In some embodiments, each of the plurality of filter chambers 1301 comprises a filter configured to receive fluid from an inlet tube 1304. In some embodiments, the rotating device 1302 may be one of a rotating cannister or a turntable. The rotating device 1302 is configured to rotate to align a first filter chamber 1301 having a first filter therein with the inlet tube 1304 to allow fluid to flow from the inlet tube 1304 to the first filter. In some embodiments, each of the plurality of filters are configured to trap at least a portion of the undesired material from the unfiltered fluid. In some embodiments, each of the plurality of filters is configured to allow fluid to flow through the plurality of filters. In some embodiments, each of the plurality of filters is operably connected to an outlet tube 1303 to allow the filtered fluid to exit the revolver filter device 1300. In some embodiments, the outlet tube 1303 is operably connected to a patient and is configured to reintroduce the filtered fluid back to the patient. In some embodiments, the outlet tube 1303 is operably connected to a reservoir or a waste receptacle.

In some embodiments, the rotating device 1302 is configured to rotate to align the inlet tube 1304 with a second filter chamber 1301 and a second filter to allow fluid to flow from the inlet tube 1304 to the second filter. In some embodiments, the rotating device 1302 is configured to rotate after the second filter chamber 1301 has reached a threshold level of captured undesired material. In some embodiments, the rotating device 1302 is configured to rotate and align each of the plurality of filter chambers 1301 with the inlet tube 1304 in a passive reinfusion system. In some embodiments, the rotating device 1302 is configured to rotate and align each of the plurality of filter chambers 1301 with the inlet tube 1304 in an active reinfusion system. In some embodiments, the rotating device 1302 is configured to rotate automatically once a respective filter has reached the threshold level of captured undesired material. In some embodiments, the rotating device 1302 is configured to rotate manually once a respective filter has reached the threshold level of captured undesired material. In some embodiments, each of the plurality of filters is configured to be replaced while the inlet tube 1304 is not aligned with the respective filter.

In some embodiments, each of the plurality of filters has the same pore size. In some embodiments, each of the plurality of filters has a different pore size. In some embodiments, each of the plurality of filters has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, or any value or range of values between any two of these values. In some embodiments, each of the plurality of filters has a pore size of about 40 μm. In some embodiments, each of the plurality of filters is configured to filter fluid to a level acceptable for reinfusion into the patient.

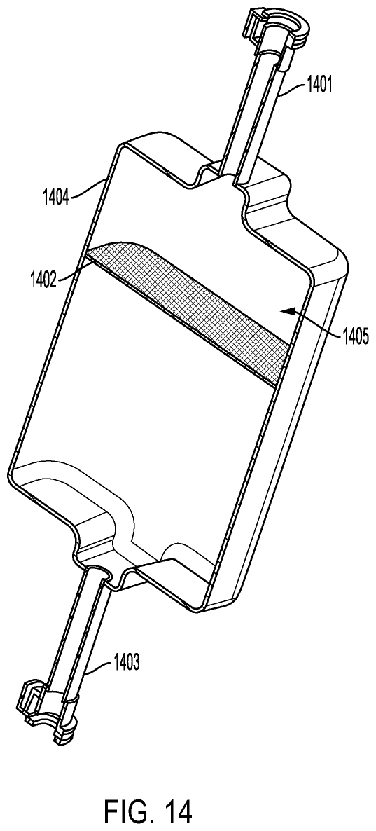

FIG. 14 illustrates a reservoir filter housing 1404 configured to hold fluid during a medical procedure. In some embodiments, the filter housing can include a waste receptacle or reservoir. The reservoir filter housing 1404 comprises an inlet port 1401 configured to receive unfiltered fluid from a pump or other source of aspiration. In some embodiments, the reservoir filter housing 1404 comprises a filter member 1402, such as a mesh or a screen, configured to filter undesired material as the undesired material passes through the reservoir filter housing 1404. In some embodiments, the reservoir filter housing 1404 comprises an outlet port 1403 configured to allow filtered fluid to flow from the reservoir filter housing 1404 to a filter system. In some embodiments, the reservoir filter housing 1404 further comprises a de-airing port configured to allow air to be removed from the reservoir filter housing 1404. In some embodiments, the de-airing port is one of a valve, a floating valve, stopcock, or a leur lock. In some embodiments, the valve is a one-way check valve.

In some embodiments, the filter member 1402 is configured to remove undesirable material from unfiltered fluid as the unfiltered fluid passes through the reservoir filter member 1402. The filter member 1402 may have a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the filter member 1402 has a pore size of about 750 to about 1000 μm.

In some embodiments, the filter member 1402 is configured to remove undesirable material, such as large bore thrombi, from the fluid. The filter member 1402 may comprise a visualization chamber 1405, defined by the space between the inlet port 1401 and filter member 1402. The removal of undesirable material or other large bore thrombi in the reservoir filter housing 1404 allows a user to visualize the undesired material removed from the patient within the visualization chamber 1405. Additionally, removal of undesirable material in the reservoir filter housing prevents the undesirable material from clogging a filter in future efforts to filter the fluid.

In some embodiments, the reservoir filter housing 1404 is transparent to allow a user to see the contents of the reservoir filter housing 1404. This allows a user to view the undesired material removed during a thrombectomy or other medical procedure. In some embodiments, the reservoir filter housing 1404 is configured to open to allow a user to remove the undesired material from the reservoir filter housing 1404.

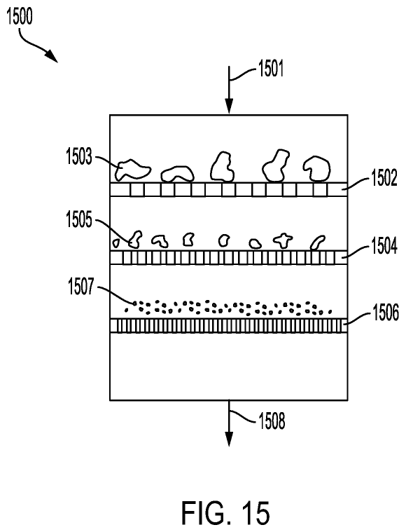

FIG. 15 illustrates a filter device 1500 with a plurality of filter sections configured to filter undesired material from fluid. The filter device 1500 comprises an inlet 1501 configured to allow fluid to flow into the filter device 1500 and an outlet 1508 configured to allow fluid to exit the filter device 1500. In some embodiments, the filter device 1500 comprises a large filter section 1502 configured to filter large pieces of undesirable material 1503, a medium filter section 1504 configured to filter medium-sized pieces of undesirable material 1505, and a small filter section 1506 configured to filter small pieces of undesirable material 1507. In some embodiments, the large filter section 1502 has a pore size of about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, or any value or range of values between any two of these values. In some embodiments, the medium filter section 1504 has a pore size of about 100 μm, about 150 μm, about 200 μm, about 250 μm, about 300 μm, about 350 μm, about 400 μm, about 450 μm, about 500 μm, or any value or range of values between any two of these values. In some embodiments, the small filter section 1506 has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values.

The filter device 1500 may have any number of filter sections configured to remove undesired material from fluid. In some embodiments, the filter device 1500 has 2, 3, 4, 5, 6, 7, 8, 9, or 10 filter sections. In some embodiments, the inlet 1501 is operably connected to an inlet tube configured to allow fluid to flow to the filter device 1500. In some embodiments, the inlet tube comprises a connection tube that is connected to a handle and/or pump. In some embodiments, the outlet 1508 is operably connected to an outlet tube configured to allow fluid to flow from the filter device 1500. In some embodiments, the outlet tube is configured to allow fluid to flow to a reservoir. In some embodiments, the outlet tube is configured to allow fluid to flow to a patient, such as via a reinfusion cannula. In some embodiments, the filter is configured to open to allow for the removal of the filtered undesired material. In some embodiments, each of the filter sections 1502, 1504, 1506 are configured to be removed and replaced by a new filter. In some embodiments, the filter is configured to filter fluid to a level acceptable for reinfusion into the patient.

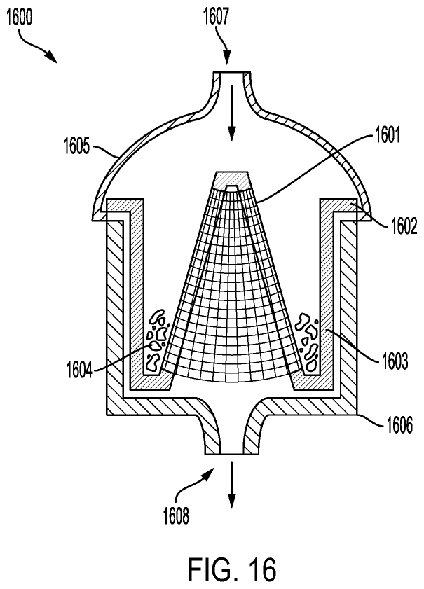

FIG. 16 illustrates a filter device 1600 configured to remove undesired material 1604 from fluid. The filter may comprise a receptacle 1603 comprising a porous cone 1601 positioned within a housing 1606. The filter may comprise an inlet 1607 configured to allow unfiltered fluid to enter the filter device 1600 and an outlet 1608 configured to allow filtered fluid to exit the filter device 1600. The filter device 1600 may further comprise a cap 1605 configured to seal the fluid within and enclose the filter device 1600. In some embodiments, the cap 1605 is configured to be removed from the filter device 1600 to allow for the removal of the undesired material. In some embodiments, the cap 1605 is configured to be removed from the filter device 1600 to allow for the replacements of the receptacle 1603. In some embodiments, the inlet is positioned on the cap 1605. The porous cone 1601 may be configured to allow unfiltered fluid to flow through the porous cone 1601 while the undesired material 1604 is held by the receptacle 1603. The conical shape of the porous cone 1601 may prevent the captured undesired material 1604 from clogging the filter device 1600 during use.

The porous cone 1601 may have pores of any size effective for the filtering of undesired material 1604 from fluid. In some embodiments, the porous cone 1601 has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the porous cone has a pore size of about 750 μm to about 1000 μm.

In some embodiments, the outer diameter of the receptacle 1603 is smaller than the inner diameter of the housing 1606 to allow the receptacle 1603 to be inserted into the housing 1606. In some embodiments, the receptacle 1603 has an interference fit with the housing 1606. In some embodiments, the receptacle 1603 has a flange 1602 configured to allow the receptacle 1603 to engage with the housing 1606 and rest on the housing 1606. In some embodiments, the inlet 1607 is operably connected to an inlet tube configured to allow fluid to flow to the filter device 1600. In some embodiments, the inlet tube comprises a connection tube that is connected to a handle and/or pump. In some embodiments, the outlet 1608 is operably connected to an outlet tube configured to allow fluid to flow from the filter device 1600. In some embodiments, the outlet tube is configured to allow fluid to flow to a reservoir. In some embodiments, the outlet tube is configured to allow fluid to flow to a patient, such as via a reinfusion cannula. In some embodiments, the filter device 1600 is configured to filter fluid to a level acceptable for reinfusion into the patient.

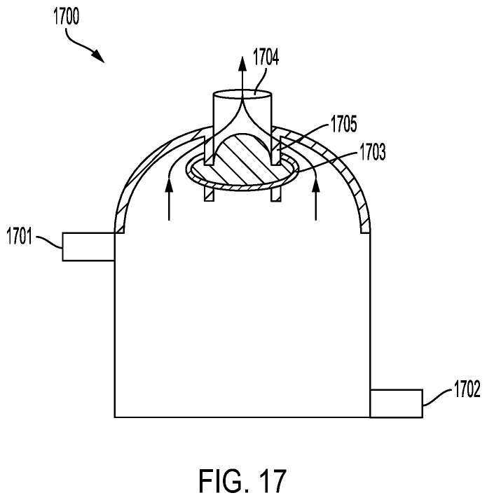

FIG. 17 illustrates a filter device 1700 configured to remove undesired material 1604 from fluid. The filter may comprise an inlet 1701 configured to allow unfiltered fluid to enter the filter device 1700 and an outlet 1702 configured to allow filtered fluid to exit the filter device 1700. The filter device 1700 may further comprise an air outlet 1704 configured to allow air to exit the filter device 1700 and a floating valve 1703 configured to seal the air outlet 1704. In some embodiments, the floating valve 1703 is configured to rest inside the filter device 1700 and allow air to flow out of the air outlet 1704. As the filter device 1700 becomes filled with fluid, the floating valve 1703 is configured to float to the top of the filter device 1700 and seal the air outlet 1704 to prevent fluid from exiting the filter device 1700 at the air outlet 1704. In some embodiments, the filter device 1700 further comprises an alignment mechanism 1705 configured to align the floating valve 1703 with the air outlet 1704 and ensure a proper seal. This may allow for pressure to be relieved from the filter device 1700 as fluid fills the filter device 1700. In some embodiments, the inlet 1701 is operably connected to an inlet tube configured to allow fluid to flow to the filter device 1700. In some embodiments, the inlet tube comprises a connection tube that is connected to a handle and/or pump. In some embodiments, the outlet 1702 is operably connected to an outlet tube configured to allow fluid to flow from the filter device 1700. In some embodiments, the outlet tube is configured to allow fluid to flow to a reservoir. In some embodiments, the outlet tube is configured to allow fluid to flow to a patient, such as via a reinfusion cannula. In some embodiments, the filter is configured to filter fluid to a level acceptable for reinfusion into the patient.

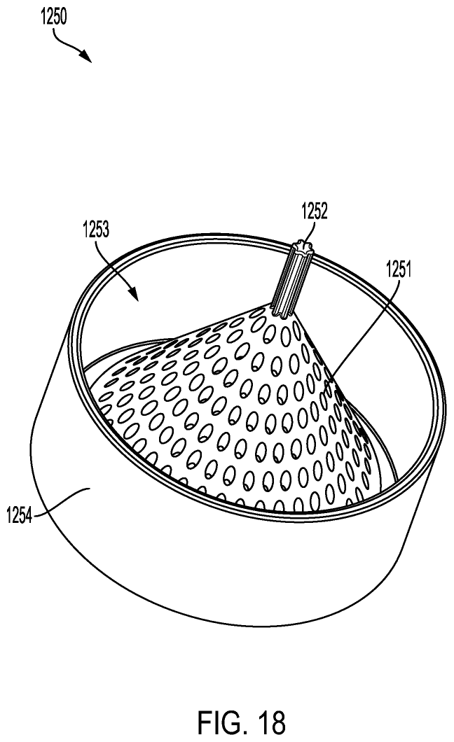

Referring now to FIG. 18, an alternative embodiment for a filter member 1250 is shown. The filter member 1250 is configured for use in a visualization device such as for example, any of the visualization device discussed herein. The filter member 1250 is configured to filter fluid within a visualization device. In some embodiments, the filter member 1250 comprises a plurality of pores 1251 configured to allow fluid to flow through the filter member 1250 and to prevent the flow of large bore thrombi or other large undesirable material through the filter member 1250. The filter member 1250 may have any pore size effective for the removal of large bore thrombi or large undesirable material from the fluid while allowing the fluid to flow through the filter member 1250. In some embodiments, the filter member 1250 has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the filter member 1250 has a pore size of about 750 μm to about 1000 μm.

In some embodiments, the filter member 1250 comprises a removal mechanism 1252 configured to allow the filter member 1250 to be removed from a visualization device. In some embodiments, the removal mechanism 1252 is one of a tab or a handle. The filter member 1250 may be any shape effective for filtering thrombi or undesirable material within a visualization device. In some embodiments, the filter member 1250 has a central porous cone 1253 surrounded by a vertically extending wall 1254 defining a receptacle for capturing the undesired material as it passes through the visualization device. Having the filter member 1250 shaped in this way prevents the captured undesired material from escaping when the user is opening the visualization device according to aspects of the disclosure described herein. In some embodiments, the surface of the filter member 1250 comprises a coating configured to reduce the adhesion of fluid to the filter member 1250 as compared to an uncoated filter member. In some embodiments, the coating is comprised of PTFE and/or any other now known or later developed anti-thrombus coating.

### Methods of Use

Methods may be performed to filter fluid using the above-described systems.

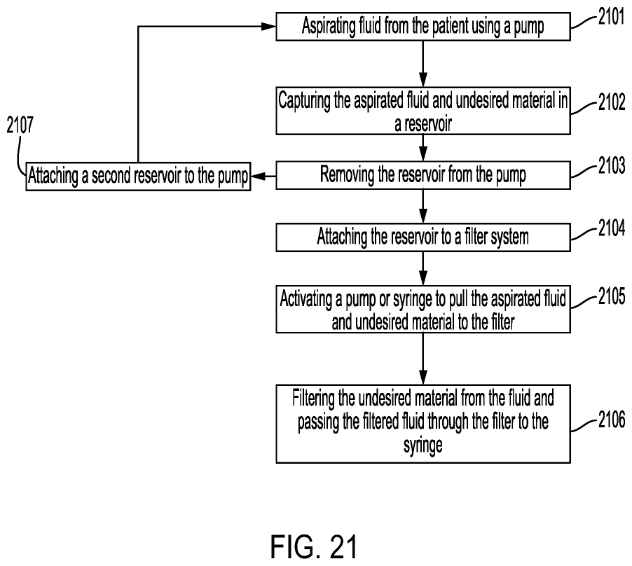

FIG. 21 depicts a flow diagram of a method of passive reinfusion and filtering fluid during a thrombectomy procedure. As show in in FIG. 21, the illustrative method comprises aspirating 2101 fluid from a patient using a pump wherein the fluid contains undesired material. The method may further comprise capturing 2102 the aspirated fluid and undesired material in a reservoir operably connected to the pump such as for example, by tubing. In some embodiments, the undesired material comprises one or more thrombi, clot, lesion, pulmonary embolism, tumors, infective vegetations, foreign bodies, or other natural objects. The method may further comprise removing and/or detaching 2103 the reservoir from the pump. In some embodiments, the method further comprises attaching 2107 a second reservoir to the pump to continue aspirating 2101 fluid and undesired material.

The method may further comprise attaching 2104 the reservoir to a filter system. In some embodiments, the filter system can include the filter system of FIG. 1 and comprise a syringe 101, a filter 102, and a fluid flow control device 103. In some embodiments, the reservoir is operably connected to the filter system using tubing 105, 106. In some embodiments, the reservoir 104 is operably connected to the filter 102, and the filter 102 is operably connected to the syringe 101.

The method may further comprise activating 2105 the pump or syringe to create suction force to pull the fluid and undesired material from the reservoir through the filter. The method may further comprise filtering 2106 the undesired material from the fluid and passing the filtered fluid through the filter to the syringe. The method may further comprise closing the fluid flow through the tubing using a fluid flow control device and detaching the syringe from the filter system. In some embodiments, the method further comprises reinfusing the filtered fluid into the patient. The method may further comprise replacing the syringe with a second syringe and continuing filtering fluid until all fluid has been removed from the reservoir. In some embodiments, the method may further comprise sealing the tubing using the fluid flow control device and removing the filter from the filter system. In some embodiments, the method may comprise replacing the filter with a second filter after the filter has reached a threshold level of captured undesired material.

The method may further comprise removing undesired material from the reservoir. In some embodiments, removing undesired material from the reservoir comprises removing the reservoir from the filter system and squeezing the undesired material out of the reservoir. In some embodiments, removing undesired material from the reservoir comprises cutting the reservoir and removing the undesired material through the cut. In some embodiments, the method further comprises sending the undesired material to a pathology lab for testing. In further embodiments, the method includes photographing and/or taking images of the undesired material to be shared with the patient, colleagues or on the user's social media page.

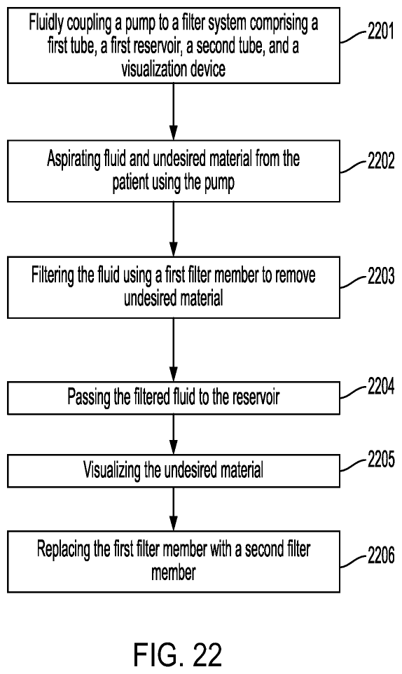

FIG. 22 depicts a flow diagram of an illustrative method of filtering fluid during a thrombectomy procedure. The method may comprise fluidly coupling 2201 a pump to a filter system comprising a first tube, a first reservoir, a second tube, and a visualization device comprising a first filter member configured to filter fluid as the fluid passes through the visualization device. The pump may be any pump effective for removing fluid and undesired material from a patient known to one of skill in the art. In some embodiments, the pump may comprise the aspiration device as described in Ser. No. 17/170,752 or may comprise any manual mechanical aspiration source such as a syringe or hand pump, or source of aspiration such as a vacuum, peristaltic pump, centrifugal pump, or any other source of aspiration known in the art.

The method may further comprise aspirating 2202 fluid and undesired material from the patient using the pump. The method may further comprise filtering 2203 the fluid using the first filter member to remove the undesired material. In some embodiments, filtering 2203 the fluid includes the removal of one or more undesirable material from the fluid. The first filter member may be configured to provide a first stage of filtration and filter 2203 undesired material of any size. In some embodiments, the first filter member has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member has a pore size of about 750 μm to about 1000 μm.

The method may further comprise passing 2204 the partially filtered fluid to the reservoir, visualizing 2205 the undesired material, and replacing 2206 the first filter member with a replacement filter member.

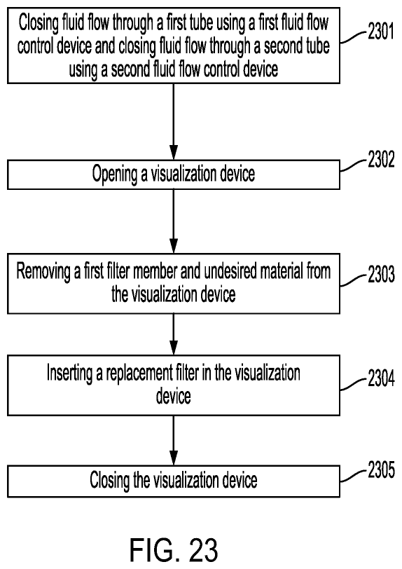

FIG. 23 depicts a flow diagram of an illustrative method of replacing 2206 the first filter member with a replacement filter member. In some embodiments, the method comprises closing 2301 fluid flow through the first tube with a first fluid flow control device, closing 2301 fluid flow through the second tube with a second fluid flow control device, and opening 2302 the visualization device. In some embodiments, replacing 2206 the first filter member further comprises removing 2303 the first filter member and the undesired material from the visualization device, inserting 2304 the replacement filter member in the visualization device, and closing 2305 the visualization device. In some embodiments, replacing 2206 the first filter member with a replacement filter member further comprises removing the visualization device from the filter system. In some embodiments, replacing 2206 the first filter member with a replacement filter member further comprises flushing the visualization device by injecting a flushing liquid into the inlet port.

The second filter member may be configured to filter 2203 undesired material of any size. In some embodiments, the replacement filter member has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the replacement filter member has a pore size of about 750 μm to about 1000 μm.

In some embodiments, the method may further comprise visualizing the filtered undesired material within the visualization device. In some embodiments, the method may comprise draining remaining fluid from the visualization device to allow for a clearer view of the undesired material within the visualization device in comparison to a visualization device that is not drained. The method may further comprise multiple methods of draining the fluid from the visualization device including, but not limited to, holding the visualization device above the first reservoir, opening a port on the visualization device, disconnecting the first tube from the visualization device, and applying pressure to the reservoir to force air out of the reservoir. In some embodiments, the visualization device may comprise an inlet port and the method further comprises flushing the visualization device by injecting a flushing liquid into the inlet port. In some embodiments, the inlet port is a luer lock. In some embodiments, the flushing liquid is saline. In some embodiments, the reservoir comprises a drain port configured to allow air and fluid to be removed from the reservoir. In some embodiments, the drain port is configured to be operably connected to a syringe or a pump to allow a user to drain air and fluid from the reservoir.

In some embodiments, the method may comprise visualizing the filtered undesired material after the filtered undesired material is removed from the visualization device. In some embodiments, a user analyzes the filtered undesired material and determines whether to perform additional aspirations for additional undesired material. In some embodiments, a user analyzes the filtered undesired material and determines whether to perform imaging to verify the amount of undesired material removed. In some embodiments, the method further comprises sending the undesired material to a pathology lab for testing.

A benefit of using a system with a visualization device is the user may determine the amount of undesired material removed which can result in less contrast being used in the patient and less imaging being performed to detect additional undesired material in the patient as compared to a method where the filtered undesired material is not visualized. Reducing the amount of contrast used in a patient is beneficial because the contrast can have a negative effect on the health of the patient's kidneys.

In some embodiments, a user analyzes the filtered undesired material and determines whether the thrombectomy procedure is complete or if the reservoir is full. In some embodiments, a user may choose to continue or terminate the procedure based on the patient's status including, but not limited to, the undesired material visualized, the patient's blood oxygenation level, pulmonary blood pressure, pulse rate, or blood pressure. In some embodiments, a user analyzes the filtered undesired material and determines whether the fluid will be returned to the patient. In some embodiments where the user determines the reservoir is full or the thrombectomy procedure is complete, the method further comprises removing the reservoir from the filter system. In some embodiments where the user determines the reservoir is full, the method further comprises sealing the second tube using a second fluid flow control device and replacing the reservoir with a second reservoir.

In some embodiments, the filter system comprises a second filter member and the method further comprises a second stage of filtration to further filter undesired material from the fluid. In some embodiments, the second filter member is positioned after the first filter member. In some embodiments, the second filter member has a pore size smaller than the pore size of the first filter member. The second filter member may be any filter effective for removing undesired material from fluid known to one of skill in the art. In some embodiments, the second filter member has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member has a pore size of about 40 μm. In some embodiments, the second filter member is configured to filter fluid to a level acceptable for reinfusion into the patient. In some embodiments, the method further comprises removing the second filter member from the filter system. In some embodiments, the method further comprises replacing the second filter member after the second filter member has filtered a threshold amount of undesired material. In some embodiments, the second filter member is positioned inside the visualization device. In some embodiments, the second filter member is configured to be replaced when the first filter member is replaced.

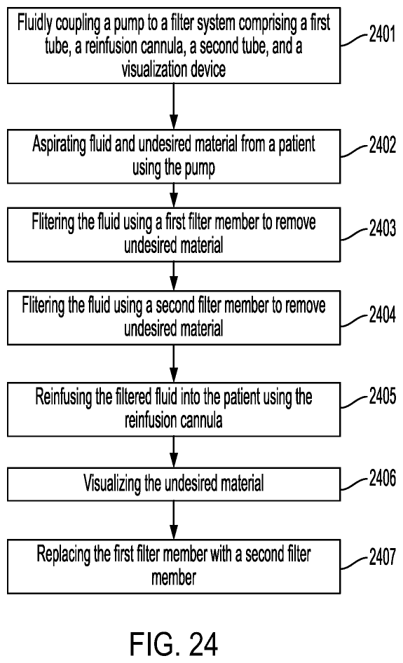

FIG. 24 depicts a diagram of a method of filtering and reinfusing fluid during a thrombectomy procedure. The method comprises fluidly coupling 2401 a pump system comprising a cannula positioned inside a patient and a pump to a filter system comprising a first tube, a reinfusion cannula, a second tube, a visualization device comprising a first filter member configured to provide a first stage of filtration to filter fluid as the fluid passes through the visualization device, and a second filter member configured to provide a second stage of filtration to further filter the partially filtered fluid as the partially filtered fluid passes through the second filter member. The pump may be any pump effective for removing fluid and undesired material from a patient known to one of skill in the art. In some embodiments, the pump may comprise the aspiration device as described in Ser. No. 17/170,752 or may comprise any manual mechanical aspiration source such as a syringe or hand pump, or source of aspiration such as a vacuum, peristaltic pump, centrifugal pump, or any other source of aspiration known in the art.

The method may further comprise aspirating 2402 fluid and undesired material from the patient using the pump. In some embodiments, the method may further comprise priming the filter system with a priming fluid prior to aspirating 2402 the fluid and the undesired material to remove all air from the filter system. In some embodiments, the filter system comprises at least one port and at least one inlet port to allow for priming the filter system. In some embodiments, the at least one port may be positioned adjacent to the reinfusion cannula. The inlet port may be positioned at any position on the system effective to introduce fluid to the system and remove air from the system including, but not limited to, the first tube, the visualization device, or the second tube. In some embodiments, the priming fluid is saline.

The method may further comprise a first filtration stage configured for filtering 2403 the fluid using the first filter member to remove the undesired material. In some embodiments, filtering 2403 the fluid includes the removal of one or more undesirable material from the fluid. The first filter member may be configured to filter 2403 undesired material of any size. In some embodiments, the first filter member has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the first filter member has a pore size of about 750 μm to about 1000 μm.

The method may further comprise a second stage of filtration configured for filtering 2404 undesired material from the partially filtered fluid using the second filter member. In some embodiments, the second filter member may be positioned after the first filter member. In some embodiments, the second filter member has a pore size smaller than the pore size of the first filter member. The second filter member may be any filter effective for removing undesired material from fluid known to one of skill in the art. In some embodiments, the second filter member has a pore size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any value or range of values between any two of these values. In some embodiments, the second filter member has a pore size of about 40 μm. In some embodiments, the second filter member is configured to filter 2404 fluid to a level acceptable for reinfusion into the patient. In some embodiments, the method further comprises removing the second filter member from the filter system. In some embodiments, the method further comprises replacing the second filter member after the second filter member has filtered 2404 a threshold amount of undesired material. In some embodiments, the second filter member is positioned inside the visualization device. In some embodiments, the second filter member is configured to be replaced when the first filter member is replaced.

The method further comprises reinfusing 2405 the filtered fluid into the patient using the reinfusion cannula. In some embodiments, reinfusing 2405 the filtered fluid into the patient comprises the pump creating a reinfusion force sufficient to drive the filtered fluid through the reinfusion cannula and back into to the body. In some embodiments reinfusing 2405 the filtered fluid into the patient comprises detaching the filter system from the pump system and holding the visualization device vertically above the reinfusion cannula to allow the fluid in the filter system to drain into the patient through the reinfusion cannula. The use of gravity to reinfuse fluid into the patient may reduce the occurrence of hemolysis as compared to pump-driven reinfusion. The reinfusion cannula may be any size effecting for reinfusing 2405 the filtered fluid into the patient. In some embodiments, the reinfusion cannula has a size of about 14 Fr, about 15 Fr, about 16 Fr, about 17 Fr, about 18 Fr, about 19 Fr, about 20 Fr, about 21 Fr, about 22 Fr, or any value or range of values between any two of these values.

In some embodiments, the method further comprises visualizing 2406 the filtered undesired material within the visualization device. In some embodiments, the visualization device comprises an inlet port and the method further comprises flushing the visualization device by injecting a flushing liquid into the inlet port. In some embodiments, the inlet port is a luer lock. In some embodiments, the flushing liquid is saline. Injecting a flushing fluid into the system allows a user to dilute the fluid in the visualization device and visualize 2406 the filtered undesired material while the filter system is primed. Visualizing 2406 the filtered undesired material allows a user to determine whether to perform additional aspirations to remove fluid and undesired material, to perform additional imaging to determine the amount of clot removed, or to terminate the procedure.

In some embodiments, the method comprises visualizing 2406 the filtered undesired material after the filtered undesired material is removed from the visualization device. In some embodiments, a user analyzes the filtered undesired material and determines whether to perform additional aspirations for additional undesired materials. In some embodiments, a user analyzes the filtered undesired material and determines whether to perform imaging to verify the amount of undesired material removed. In some embodiments, the method further comprises sending the undesired material to a pathology lab for testing.

The method further comprises replacing 2407 the first filter member with a replacement filter member. In some embodiments, replacing 2407 the first filter member with a replacement filter member comprises sealing the first tube with a first fluid flow control device, sealing the second tube with a second fluid flow control device, and opening the visualization device. In some embodiments replacing 2407 the first filter member further comprises removing the first filter member and the undesired material from the visualization device, inserting the replacement filter member in the visualization device, and closing the visualization device. In some embodiments, replacing 2407 the first filter member with a replacement filter member further comprises removing the visualization device from the filter system. In some embodiments, replacing 2407 the first filter member with a replacement filter member further comprises flushing the visualization device by injecting a flushing liquid into the inlet port. In some embodiments, replacing 2407 the first filter member comprises removing the second filter member from the filter system and replacing the second filter member. In some embodiments, the method further comprises priming the filter system with a priming fluid to remove air from the filter system.

The replacement filter member may be configured to filter 2403 undesired material of any size. In some embodiments, the replacement filter member has a pore size of about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm, or any value or range of values between any two of these values. In some embodiments, the replacement filter member has a pore size of about 750 μm to about 1000 μm.

A benefit of using a system with a visualization device is the user may determine the amount of undesired material removed which can result in less contrast being used in the patient and less imaging being performed to detect undesired material in the patient as compared to a method where the filtered undesired material is not visualized. Reducing the amount of contrast used in a patient is beneficial because the contrast can have a negative effect on the health of the patient's kidneys. In some embodiments, a user analyzes the filtered undesired material and determines whether the thrombectomy procedure is complete or if the reservoir is full. In some embodiments, a user may determine to continue or terminate the procedure based on the patient's status including, but not limited to, the patient's blood oxygenation level, pulmonary blood pressure, pulse rate, or blood pressure. In some embodiments, a user analyzes the filtered undesired material and determines whether the fluid will be returned to the patient. In some embodiments where the user determines the reservoir is full or the thrombectomy procedure is complete, the method further comprises removing the reservoir from the filter system. In some embodiments where the user determines the reservoir is full, the method further comprises sealing the second tube using a second fluid flow control device and replacing the reservoir with a second reservoir.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of filtering fluid from a patient, the method comprising:
fluidly coupling a pump to a filter system comprising a first tube, a first reservoir, a second tube, and a visualization device comprising a first filter member;
aspirating fluid from the patient using the pump wherein the fluid contains an undesired material;
filtering the fluid using the first filter member to capture at least a portion of the undesired material;
passing the filtered fluid to the reservoir;
visualizing the undesirable material from the first filter member; and
replacing the first filter member with a replacement filter member.

2. The method of claim 1, wherein replacing the first filter member with a replacement filter member
sealing the first tube using a first fluid flow control device and sealing the second tube using a second fluid flow control device;
opening the visualization device;
removing the first filter member and the captured portion of the undesired material from the visualization device;
inserting the replacement filter member in the visualization device; and
closing the visualization device.

3. The method of claim 2, wherein the visualization device comprises a first flushing port, the method further comprising:
flushing the visualization device by injecting a flushing liquid into the first flushing port.

4. The method of claim 1, wherein at least one of the first filter member and the replacement filter member has a pore size of about 750 μm to about 1000 μm.

5. The method of claim 1, wherein at least one of the first filter member and the replacement filter member has a conical shape.

6. The method of claim 1, wherein at least one of the first filter member and the replacement filter member has a tab positioned on a top surface of the first filter member and the replacement filter member.

7. The method of claim 1, wherein the filter system comprises a second filter member positioned downstream of the first filter member, the method further comprising filtering the fluid using the second filter member to capture the undesired material.

8. The method of claim 7, wherein the second filter member has a pore size of about 40 μm.

9. The method of claim 7, wherein the second filter member is configured to be replaced.

10. The method of claim 7, wherein the second filter member is positioned inside the visualization device.

11. The method of claim 1, further comprising:
sealing the second tube using a second fluid flow control device; and
replacing the first reservoir with a second reservoir.

12. A method of filtering fluid from a patient, the method comprising:
fluidly coupling a pump to a filter system comprising a first tube, a reinfusion cannula, a second tube, a visualization device comprising a first filter member, and a second filter member;
aspirating fluid from the patient using the pump wherein the fluid contains an undesired material;
filtering the fluid using the first filter member to capture at least a first portion of undesired material;
filtering the fluid using the second filter member to capture a second portion of the undesired material, thereby generating a filtered fluid;
reinfusing the filtered fluid into the patient using the reinfusion cannula;
visualizing the undesirable material from the first filter member; and
replacing the first filter member with a replacement filter member.

13. The method of claim 12, wherein the filter system comprises a valve and replacing the first filter member with a replacement filter member comprises:
sealing the first tube using a first fluid flow control device and sealing the second tube using a second fluid flow control device;
opening the visualization device;
removing the first filter member and the first portion of the undesired material from the visualization device;
inserting the replacement filter member in the visualization device;
closing the visualization device; and,
priming the filter system with a priming fluid to remove air from the filter system.

14. The method of claim 13, wherein the first fluid flow control device is one of a clamp or a stopcock.

15. The method of claim 12, wherein the visualization device comprises a first flushing port, the method further comprising:
flushing the visualization device by injecting a flushing liquid into the first flushing port.

16. The method of claim 15, wherein the flushing liquid is saline.

17. The method of claim 12, wherein at least one of the first filter member and the replacement filter member has a conical shape.

18. The method of claim 12, wherein at least one of the first filter member and the replacement filter member has a tab positioned on a top surface of the first filter member and the replacement filter member.

19. The method of claim 12, wherein the second filter member is configured to be replaced.

20. The method of claim 12, wherein the second filter member is positioned inside the visualization device.
